(19) United States
(12) Patent Application Publication
(10) Pub. No.: US 2005/0250677 A1
(43) Pub. Date: Nov. 10, 2005

(54) Glycopeptide antibiotic derivatives

(76) Inventors: Jan Balzarini, Heverlee (BE); Maria Preobrazhenskaya, Moscow (RU); Erik De Clercq, Lovenjoel (BE)

Correspondence Address: CLARK & ELBING LLP, 101 FEDERAL STREET, BOSTON, MA, 02110, US

(21) Appl. No.: 10/525,784

(22) PCT Filed: Sep. 1, 2003
(86) PCT No.: PCT/BE03/00144

§ 371 Date: Feb. 28, 2005

(30) Foreign Application Priority Data
Aug. 30, 2002 (GB) 0220235.6
Aug. 31, 2002 (GB) 020233.1
Apr. 25, 2003 (GB) 0310890.9
Apr. 25, 2003 (GB) 0309521.3

(51) Int. Cl. A61K 38/14; C07K 9/00

(52) U.S. Cl. 514/8; 530/322

(57) ABSTRACT

Novel glycopeptide antibiotic derivatives, processes for their preparation, their use as a medicine, their use to treat or prevent viral infections and their use to manufacture a medicine to treat or prevent viral infections are provided. The present invention relates to the use of glycopeptide antibiotics and their semisynthetic derivatives to treat or prevent viral infections and their use to manufacture a medicine to treat or prevent viral infections of subjects, more in particular infections with viruses belonging to Retroviridae, Herpes viridae, Flaviviridae and the Coronaviridae, like HIV (human immunodeficiency virus), HCV (hepatitis C virus), BVDV (bovine viral diarrhoea virus), SARS (severe acute respiratory syndrome) causing virus, FCV (feline coronavirus), HSV (herpes simplex virus), VZV (varicella zoster virus) and CMV (cytomegalovirus).

### CROSS REFERENCE TO RELATED APPLICATIONS

This application is the U.S. National Stage of International Application No. PCT/BE2003/000144, filed Sep. 1, 2003, which claims the benefit of GB 0220235.6, GB 0220233.1, GB 0310890.9, and GB 0309521.3, filed Aug. 30, 2002, Aug. 31, 2002, Apr. 25, 2003, and Apr. 25, 2003, respectively.

### FIELD OF THE INVENTION

The field of the invention relates to novel glycopeptide antibiotic derivatives, processes for their preparation, their use as a medicine, their use to treat or prevent viral infections and their use to manufacture a medicine to treat or prevent viral infections. The present invention relates to the use of glycopeptide antibiotics and their semisynthetic derivatives to treat or prevent viral infections and their use to manufacture a medicine to treat or prevent viral infections of subjects, more in particular infections with viruses belonging to Retroviridae (i.e. Lentivirinae), Herpes viridae, Flaviviridae and the Coronaviridae, like HIV (human immunodeficiency virus), HCV (hepatitis C virus), BVDV (bovine viral diarrhoea virus), SARS (severe acute respiratory syndrome) causing virus, FCV (feline coronavirus), HSV (herpes simplex virus), VZV (varicella zoster virus) and CMV (cytomegalovirus).

### BACKGROUND OF THE INVENTION

Viral infections remain a major medical problem worldwide because of a lack of therapy, prevention or vaccination strategy and because of the rapid development of resistance. Viruses can be devided into two big groups, RNA-viruses and DNA-viruses, according to their genetic composition, which can then further be subdivided. Human pathogens include Adenovirus, Cytomegalovirus, Dengue virus, Ebola virus, Enterovirus, Epstein Bar Virus, Hantavirus, Hepatitis A virus, Hepatitis B virus, Hepatitis C virus, Herpes Simplex virus, Human Herpes Virus 8, Human Immunodeficiency Virus, Human Metapneumovirus, Human Papilloma Virus, Influenza virus, La Crosse Virus, Marburg virus, Nipah virus, Parvovirus B19, Polyoma BK virus, Polyoma JC virus, Respiratory Syncytial Virus, Variola, Coxsackie virus and others.

HIV-I (human immunodeficiency virus-1) is one of these problematic viral infections with an estimated 40 million people infected worldwide. There are several strains of HIV. The two main ones are HIV-1 and HIV-2, the latter one producing a less severe disease than the first one. The number of cases of HIV and AIDS (acquired immunodeficiency syndrome) has risen rapidly. In 1999, 5.6 million new infections were reported, and 2.6 million people died from AIDS. Currently available drugs for the treatment of HIV include nucleoside reverse transcriptase (RT) inhibitors (i.e. zidovudine, didanosine, stavudine, lamivudine, zalcitabine and abacavir), non-nucleoside reverse transcriptase inhibitors (i.e. nevirapine, delavirdine and efavirenz), peptidomimetic protease inhibitors (i.e. saquinavir, indinavir, ritonavir, nelfinavir, amprenavir and lopinavir) and the entry inhibitor enfuvirtide. A relatively new target that is focussed on lately is the integrase enzyme of HIV, while also many other proteins acting as enzymes or co-factors are being investigated. Each of the currently available drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients ultimately fail combination drug therapies. Insufficient drug potency, noncompliance, restricted tissue penetration and drug-specific limitations within certain cell types (e.g. some nucleoside analogs cannot be efficiently phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present.

Many other virusses and virus families causing problematic disorders can be identified. The family of the Flaviviridae for example consists of 3 genera, the pestiviruses, the flaviviruses (i.e. Dengue virus) and the hepaciviruses (also containing the hepatitis G virus (HGV/GBV-C) that has not yet been assigned to a genus) which can be responsible for severe diseases. Pestiviruses such as the Classical Swine Fever Virus (CSFV), the Bovine Viral Diarrhea Virus (BVDV) and the Border Disease Virus (BDV) cause infections of domestic livestock (respectively pigs, cattle and sheep) and are responsible for significant economic losses world-wide. BVDV, the prototypic representative of the pestivirus genus is ubiquitous and causes a range of clinical manifestations, including abortion, teratogenesis, respiratory problems, chronic wasting disease, immune system dysfunction, and predisposition to secondary viral and bacterial infections and may also cause acute fatal disease. Foetuses of cattle can be infected persistently with BVDV, these animals remain viremic throughout life and serve as continuous sources for virus spread in herds. Vaccines are used in some countries with varying degrees of success to control pestivirus disease (Leyssen P, et al., Clin Microbiol Rev. 2000 January; 13(1):67-82).

The World Health Organization estimates that world-wide 170 million people (3% of the world's population) are chronically infected with HCV (Leyssen P, et al., Clin Microbiol Rev. 2000 January; 13(1):67-82). These chronic carriers are at risk of developing cirrhosis and/or liver cancer. In studies with a 10 to 20 year follow-up, cirrhosis developed in 20-30% of the patients, 1 to 5% of whom may develop liver cancer during the next ten years (Dutta et al, Hum. Pathol. 1998 November; 29(11):1279-84). The only treatment option available today is the use of interferon α-2 (or its pegylated from) either alone or combined with ribavirin. However, sustained response is only observed in about 40% of the patients and treatment is associated with serious adverse effects (reviewed in Leyssen et al., 2000). There is thus an urgent need for potent and selective inhibitors of the replication of HCV in order to treat infections with HCV. Furthermore, the study of specific inhibitors of HCV replication has been hampered by the fact that it is not possible to propagate HCV (efficiently) in cell culture. Since HCV and pestiviruses belong to the same virus family and share many similarities (organisation of the genome, analogous gene products and replication cycle), pestiviruses have been adopted as a model and surrogate for HCV. For example BVDV is closely related to hepatitis C virus (HCV) and used as a surrogate virus in drug development for HCV infection (Zitzmann N. et al., Proc. Natl. Acad. Sci. USA, 96, 11878-11882 and Bukhtiyarova,-M et al., Antiviral Chem. Chemother. 2001 November; 12(6): 367-73). One compound VP32947 or (3-[((2-dipropylamino)ethyl)thio]-5H-1,2,4-triazino[5,6-b]indole has been reported to selectively inhibit the replication of BVDV and other pestiviruses (Baginski S G et al., Proc. Natl. Acad. Sci. U.S.A. 2000 Jul. 5; 97(14):7981-6). Currently, there is no treatment strategy available for controlling infections caused by pestiviruses.

The genus of the Flavivirusses comprises the pathogens Dengue virus, Yellow Fever virus and the West Nile virus which are causing major health problems worlwide (Asia, Africa, America) and for which currently no therapy is available.

The family of the Herpesviridae includes important human pathogens like Herpes simplex virus (HSV) type 1 and 2, Herpes Zoster virus (VZV), Cytomegalovirus (CMV), Epstein Bar virus (EBV) and human Herpes virus type 6 and 8 (i.e. HHV-6 and -8). These viruses cause disorders like Herpes Labialis, Herpes Genitalis, Herpes Encephalitis, Kaposi-sarcoma, Varicella, Zona, lymfomas and others. Current treatments consist of Vidarabine, Acyclovir, Gancyclovir, Brivudin, Cidofovir and some other products.

Coronaviridae now approximately comprises 15 species, which infect not only man but also cattle, pigs, rodents, cats, dogs and birds (some are serious veterinary pathogens, especially chickens and cats). Coronavirus infection is very common and occurs worldwide. The incidence of infection is strongly seasonal, with the greatest incidence in children in winter. In humans, they cause respiratory infections (including Severe Acute Respiratory Syndrome (SARS), enteric infections and rarely neurological syndromes. SARS is a form of viral pneumonia where infection encompasses the lower respiratory tract. The true cause appears to be a novel coronavirus with some unusual properties. The SARS virus can be grown in Vero cells, a novel property for Human Coronavirusses, most of which cannot be cultivated. In these cells, virus infection results in a cytopathic effect, and budding of coronavirus-like particles from the endoplasmic reticulum within infected cells (Zhang et al, Acta Bioch. Bioph. Sinica 2003, 35, 587-591). There is currently no antiviral drug available that has been shown to be consistently successful in treating SARS or any coronavirus infection, nor is there any vaccine against SARS.

As a conclusion, for many pathogenic viral infections, no efficient treatment is currently available and moreover, the available anti-viral therapies or preventive measures are not sufficient in order to able to cure, prevent or ameliorate the respective viral infections due to many reasons, like the occurence of resistance and unfavorable pharmacokinetic or safety profiles.

Therefore, there is still a stringent need in the art for potent inhibitors of viruses, such as HIV, HCV, SARS-causing virus, CMV, Herpes virusses, etc. Therefore a goal of the present invention is to satisfy this urgent need by identifying efficient and non-harmful pharmaceutically active ingredients and combination of ingredients for the treatment of viral infections in mammals and in humans. In the case of HIV for example, there is still a need for compounds which either complement existing drugs such that the resulting cocktail has improved drug resistance suppression or compounds which are themselves effective against a virus, including many or all viable mutations of a virus.

The glycopeptide, or vancomycin, class of antibiotics consists of compounds of relatively high molecular weight. Structurally, they comprise a polypeptide core aglycone structure having phenolic amino acids and one or more peripheral carbohydrate moieties (Williams et al., Topics in Antibiotic Chemistry, Volume 5, pages 119-158). Known members of this class include vancomycin (McCormick et al., U.S. Pat. No. 3,067,099), ristocetin (Philip et al., U.S. Pat. No. 2,990,329), A35512 (Michel et al., U.S. Pat. No. 4,083,964), avoparcin (Kunstmann et al., U.S. Pat. No. 3,338,786) teicoplanin (Bardone et al., J. Antibiot., Volume 31, page 170, 1978), actaplanin (Raun, U.S. Pat. No. 3,816,618), AAD-216 (Bowie et al., EP-A No. 132118), A477 (Raun et al., U.S. Pat. No. 3,928,571), OA7633 (Nishida et al., U.S. Pat. No. 4,378,348), AM 374 (Kunstmann et al., U.S. Pat. No. 3,803,306), K288 (J. Antibiotics, Series A, Volume 14, page 141 (1961), also known as actinoidin), ristomycin and others.

Some glycopeptide antibiotics, such as vancomycin and teicoplanin are vital therapeutic agents used world-wide for the treatment of infections with gram-positive bacteria. Other antibiotics of this type (eremomycin, chloroeremomycin, ristocetin, teicoplanin aglycon and some others) are also highly active against gram-positive microorganisms including methicillin-resistant staphylococci (Nagarajan, R. Glycopeptide Antibiotics. New york: Marcel Dekker. 1994). In addition, many have been demonstrated to increase animal feed utilization efficiency and, therefore, to be useful to promote animal growth, to improve milk production in ruminants and to treat and to prevent ketosis in ruminants. The glycopeptide antibiotics are well known as powerful antibacterial but until now there are no data available about anti-viral, anti-retroviral or anti-HIV activity of such compounds.

Emerging bacterial resistance to vancomycin, which has recently become a major public health threat, is a stimulus for the synthesis and investigation of various derivatives of glycopeptide antibiotics (Malabarba, A et al Med. Res. Rev. 17: 69-137, 1997 and Pavlov A. Y. & M. N. Preobrazhenskaya. Russian Journal of Bioorganic Chemistry. 24:570-587, 1998). EP00265071 and WO00/69893 for example describe novel glycopeptide antibiotics related to vancomycin with antibacterial activity. However, none of these compounds or their derivatives have been demonstrated to have antiviral properties or to be suitable to inhibit or prevent viral infections.

Several natural peptide antibiotics such as complestatins and chloropeptins with activity against HIV-1 (K. Matsuzara, H. et al J. Antibiotics 1994, V. 47, N. 10, p. 1173-1174) and kistamycins with activity against influenza virus (N. Naruse, O, et al J. Antibiotics 1993, V. 46, N. 12, p. 1812-1818) have been described. However, the structures of these hexa- or heptapeptide antibiotics and the structures of glycopeptide antibiotics and of the aglycons of glycopeptide antibiotics differ greatly in both amino acid sequence and stereochemistry. All kystamycins, complestatin and chloropeptins contain a tryptophan moiety linked to central amino acid No 4, whereas it is represented by a substituted phenylalanine moiety in vancomycin, eremomycin, chloreremomycin, teicoplanin, DA-40926 and other antibacterial glycopeptides.

Synthesis methods for glycopeptide antibiotic derivatives have also already been described as in Miroshnikova, O. V. et al. Modification of the N-Terminal Amino Acid in the Eremomycin Aglycone. J. Antibiot. 1996, 49, 1157-1161 and in Malabarba, A. et al Structural modifications of the active site in teicoplanin and related glycopeptides or Deglucoteicoplanin-derived tetrapeptide. J. Org. Chem. 1996, 61, 2151-2157) and in Malabarba, A. et al. Structural Modifications of Glycopeptide Antibiotics. Med. Res. Rev. 1997, 17, 69-137 and in Pavlov, A. Y.; Preobrazhenskaya, M. N. Chemical Modification of Glycopeptide Antibiotics. Russian Journal of Bioorganic Chemistry 1998, 24, 570-587.

Within the present invention, new anti-viral compounds have been obtained that are active against a wide range of viruses belonging to different families.

### SUMMARY OF THE INVENTION

In the present invention, new selective anti-viral compounds are being provided. The compounds are glycopeptide antibiotics from natural resources and their semisynthetic analogs and derivatives and it has been shown that they possess a broad anti-viral activity. Members of the Retroviridae (i.e. Lentivirinae), Flaviviridae, Herpesviridae and of the Coronaviridae families are being inhibited. The present invention demonstrates that the compounds inhibit the replication of BVDV, HIV, HSV, CMV, VZV, FCV and the SARS virus. Furthermore, the anti-HIV activity of the compounds is based on an activity in a early stage of the HIV infection cycle and are potential entry-inhibitors. Therefore, these glycopeptide antibiotics and their semisynthetic derivatives constitute a new potent class of anti-viral compounds that can be used in the treatment and prevention of viral infections in animals, mammals and humans, more specifically for the treatment and prevention of BVDV, HCV, HIV, CMV, FCV, SARS virus, HSV and VZV infections.

The present invention relates to glycopeptide antibiotics from natural resources or semisynthetically prepared. The present invention also relates to semisynthetic glycopeptide antibiotic derivatives. The invention further relates to compounds having anti-viral activity, more specifically to glycopeptide antibiotics and derivatives that inhibit the replication of viruses. Most particularly, the invention relates to glycopeptide antibiotics and derivatives which inhibit the replication of viruses of the family of the retroviridae (i.e. Lentivirinae), Flaviviridae, Herpesviridae and Coronaviridae and yet more specifically to compounds that inhibit the replication of BVDV (Bovine Viral Diarrhea Virus), HIV (human immunodeficiency virus), Herpes virus infections like HSV (herpes simplex virus), Varizella Zoster virus (VZV) infections, Cytomegalovirus (CMV), Feline corona virus (FCV) and the virus causing Severe acute Respiratory Syndrome (SARS). Present invention furthermore relates to the use of the compounds as a medicine and more specifically to use the compounds as an anti-viral. The invention also relates to methods for preparation of all such compounds and pharmaceutical compositions comprising them. The invention further relates to methods of structurally modifying said compounds for increasing the antiviral activity and methods of structurally modifying said compounds for decreasing or removing antibacterial activity while maintaining antiviral activity. The invention further relates to the use of said compounds in the manufacture of a medicament useful for the treatment of viral infections, more in particular of BVDV, HCV, HIV, FCV, HSV, CMV, VZV infections and infections of the virus causing SARS, as well as for treatment of other retroviral, lentiviral and viral infections. The present invention also relates to a method of treatment of viral infections, by using said compounds.

The present invention relates thus to glycopeptide antibiotics and their derivatives, including various semisynthetic derivatives of natural glycopeptide antibiotics such as vancomycin, eremomycin, chloreremomycin, teicoplanin, Deacyl-40926, Demannosyl-DA40926, ristocetin, A35512, avoparcin, actaplanin, AAD-216, A477, OA7633, AM 374, actinoidin, ristomycin and others, their aglycons and also products of their partial degradation with the peptide core destroyed or modified in peptide core and in sugar moieties. The present derivatives are useful as anti-viral compounds.

According to a first aspect, the invention relates to the use of glycopeptide antibiotics and their derivatives as antiviral compounds, more particularly as compounds active against BVDV, HCV, HIV, FCV, HSV, CMV, VZV infections and infections of the virus causing SARS. The present invention relates also to the use of glycopeptide antibiotics and their derivatives for the manufacture of a medicament useful for the treatment or prevention of viral infections.

According to a second aspect, the invention relates to glycopeptide antibiotic derivatives or in general compounds, which according to the general embodiment of the invention correspond to compounds according to the general formula Z, pharmaceutically acceptable salts, solvates, tautomers and isomers thereof, 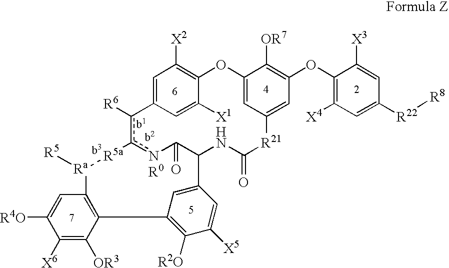
wherein,
R²¹ and R²² are taken together into a group of the formula CHNH(CO)(CH₂)ₙCHR¹NH(CO)RCH or in a group of formula A, or in the case R²¹ and R²² are not taken together, R²¹ represents R and R²² represents —R^{c}R^{5c}; 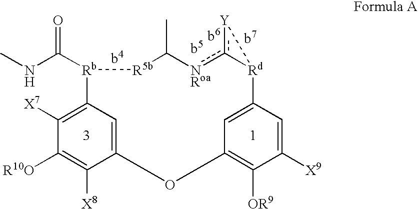
each b¹ and b² independently represents nihil or an additional bond, while b¹ and b² can not be an additional bond at the same time, R⁰ represents nihil when b² represents an additional bond and hydrogen when b² represents nihil, R⁶ represents nihil when b¹ represents an additional bond and hydrogen when b¹ represents nihil, R⁶ represents R^{6a} and R⁰ represents hydrogen when b¹ and b² each represents nihil;
b³ represents nihil or an additional bond, R^{a}—R^{5a} represents a group of the formula CHN(R¹¹)CO, CHN(R¹¹)(CH₂)_{z}N(R^{11a})CO or CHN(R¹¹)CO(CH₂)_{z}N(R^{11a})CO when b³ represents an additional bond, and R^{a} is R and R^{5a} is R⁵ when b³ represents nihil, wherein z is 0, 1, 2, 3 or 4;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R^{11a})CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and R^{d} represents R or a group of the formula (CH₂)_{q}CON(R¹¹)CH(CH₂OH) (CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R¹¹) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen, halogen and X⁶;
X⁶ is selected from the group comprising hydrogen, halogen, SO₃H, OH, NO, NO₂, NHNH₂, NHN═CHR¹¹, N═NR¹¹, CHR¹¹R¹³, CH₂N(R³)R¹¹, R⁵, R¹¹ and R¹³, wherein ³ is CH₂ attached to the phenolic hydroxyl group of the 7^{th} amino acid;
X⁸ is selected from hydrogen and alkyl;
R^{c} represents R and R^{5c} represents R⁵;
R is selected from CHR¹³ and R¹⁴;
R¹ is selected from hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)—[O-phenyl(o-OR⁹, m-X⁹, m-R¹⁶)]-m, where t is 0, 1, 2, 3 or 4;
each R² and R⁴ are independently selected from hydrogen, R¹² and R¹⁷;
R³ is selected from hydrogen, R¹², R¹⁷ and Sug;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁷ is selected from hydrogen, R¹², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug.
R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁹ is selected from hydrogen, R¹², R¹⁷ or Sug;
R¹⁰ is selected from hydrogen, R¹², R¹⁷ or Sug, wherein Sug is any cyclic or acyclic carbohydrate;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl, a heterocyclic ring, alkylphosphonate (e.g. alkylenePO₂OH) and alkylphosphonamide unsubstituted or substituted at the amide with alkyl, alkenyl or alkynyl(e.g alkylenePO₂NH₂), wherein each alkyl, alkylene, alkenyl, alkynyl, aryl, arylalkyl, heteroaryl, cyloalkyl, cycloalkenyl, cycloalkynyl and heterocyclic ring can be substituted with 1 or more R¹⁹ or Sug;
each R¹² and R^{12a} are independently selected from the group consisting of hydrogen, acyl, amino-protecting group, carbamoyl, thiocarbamoyl, SO₂R¹¹, S(O)R¹¹, COR¹³—R¹⁸, COCHR¹⁸N(NO)R¹¹, COCHR¹⁸NR¹¹R¹² and COCHR¹⁸N⁺R¹¹R^{11a}R^{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹³ is selected from the group consisting of hydrogen, NHR^{12a}, NR¹¹R¹², NR¹¹Sug, N⁺R¹¹R^{11a}R^{11b}, R¹⁵, NR¹¹C(R^{11a}R^{11b})COR¹⁵ and group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R¹¹R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R¹²;
R¹⁵ is selected from N(R¹¹)NR^{11a}R¹², N(R¹¹)OR^{11a}, NR¹¹C(R^{11a}R^{11b})COR¹³;
R¹⁶ is selected from a group of the formula R—R⁵ or CH(NH₂)CH₂OH;
R¹⁷ is selected from SO₃H, SiR¹¹R^{11a}R_{11b}, SiOR¹¹OR^{11a}OR^{11b}, PR¹¹R^{11a}, P(O)R¹¹R^{11a}, P⁺R¹¹R^{11a}R^{11b};
R¹⁸ is selected from hydrogen, R¹, alkyl, aryl, phenyl-rhamnose-p, phenyl-(rhamnose-galactose)-p, phenyl-(galactose-galactose)-p, phenyl-O-methylrhamnose-p, wherein each alkyl and aryl can be substituted with 1 or more R¹⁹ or Sug,
R¹⁹ is selected from hydrogen, halogen, SH, SR²⁰, OH, OR²⁰, COOH, COR²⁰, COOR²⁰NO₂, NH₂, N(R²⁰)₂NHC(NH₂)═NH, CH(NH₂)═NH, NHOH, NHNH₂, N₃, NO, CN, N═NR², N═NR¹², SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰.
R²⁰ is selected from hydrogen, halogen, SH, OH, COOH, NO₂, NH₂, NHC(NH₂)═NH CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring;
   and to their use as antiviral compounds and for the manufacture of a medicament to treat or prevent viral infections.

According to a particular embodiment of the second aspect, the invention relates to glycopeptide antibiotic derivatives or in general compounds, which according to the general embodiment of the invention correspond to compounds according to the general formula I, II and III, pharmaceutically acceptable salts, solvates, tautomers and isomers thereof, 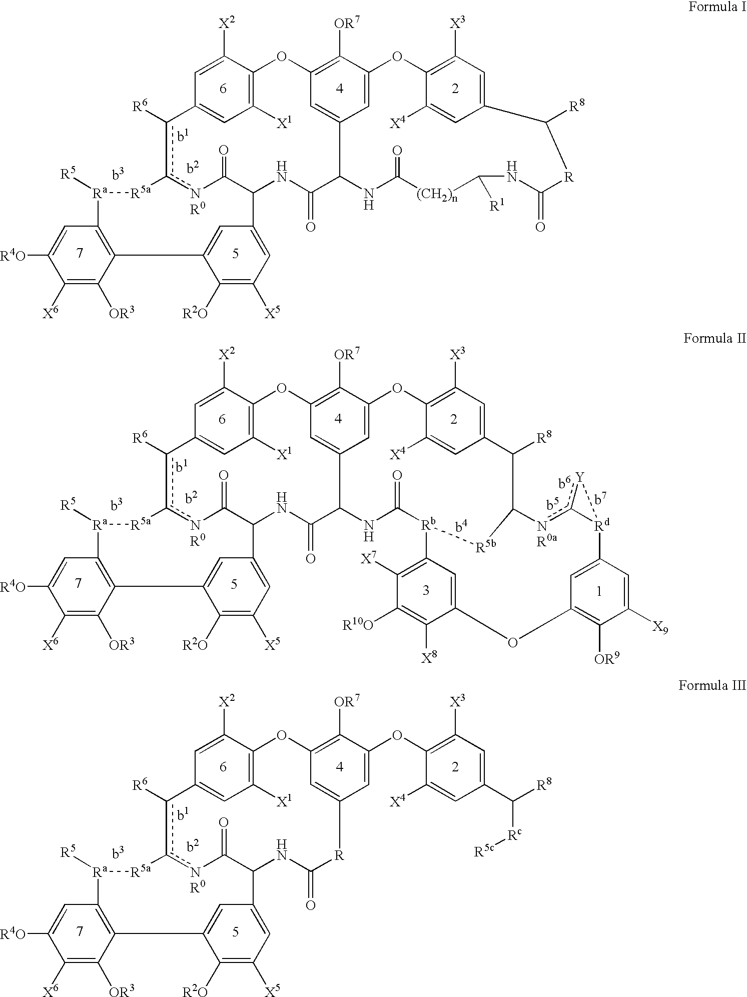
wherein:
each b¹ and b² independently represents nihil or an additional bond, while b¹ and b² can not be an additional bond at the same time, R⁰ represents nihil when b² represents an additional bond and hydrogen when b² represents nihil, R⁶ represents nihil when b¹ represents an additional bond and hydrogen when b¹ represents nihil, R⁶ represents R^{6a} and R⁰ represents hydrogen when b¹ and b² each represents nihil;
b³ represents nihil or an additional bond, R^{a}—R^{5a} represents a group of the formula CHN(R¹¹)CO, CHN(R¹¹)(CH₂)_{z}N(R^{11a})CO or CHN(R¹¹)CO(CH₂)_{z}N(R^{11a})CO when b³ represents an additional bond, and R^{a} is R and R^{5a} is R⁵ when b³ represents nihil, wherein z is 0, 1, 2, 3 or 4;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R^{11a})CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and R^{d} represents R or a group of the formula (CH₂)_{q}CON(R¹¹)CH(CH₂OH)(CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R¹¹) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen, halogen and X⁶;
X⁶ is selected from the group comprising hydrogen, halogen, SO₃H, OH, NO, NO₂, NHNH₂, NHN═CHR¹¹, N═NR¹¹, CHR¹¹R¹³, CH₂N(R³)R¹¹, R⁵, R¹¹ and R³, wherein R³ is CH₂ attached to the phenolic hydroxyl group of the 7^{th} amino acid;
X⁸ is selected from hydrogen and alkyl;
R^{c} represents R and R^{5c} represents R⁵;
R is selected from CHR¹³ and R¹⁴;
R¹ is selected from hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)—[O-phenyl(o-OR⁹, m-X⁹, m-R¹⁶)]-m, where t is 0, 1, 2, 3 or 4;
each R² and R⁴ are independently selected from hydrogen, R¹² and R¹⁷;
R³ is selected from hydrogen, R¹², R¹⁷ and Sug;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁷ is selected from hydrogen, R¹², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug.
R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁹ is selected from hydrogen, R¹², R¹⁷ or Sug;
R¹⁰ is selected from hydrogen, R¹², R¹⁷ or Sug, wherein Sug is any cyclic or acyclic carbohydrate;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl, a heterocyclic ring, alkylphosphonate (e.g. alkylenePO₂OH) and alkylphosphonamide unsubstituted or substituted at the amide with alkyl, alkenyl or alkynyl(e.g alkylenePO₂NH₂), wherein each alkyl, alkylene, alkenyl, alkynyl, aryl, arylalkyl, heteroaryl, cyloalkyl, cycloalkenyl, cycloalkynyl and heterocyclic ring can be substituted with 1 or more R¹⁹ or Sug;
each R¹² and R^{12a} are independently selected from the group consisting of hydrogen, acyl, amino-protecting group, carbamoyl, thiocarbamoyl, SO₂R¹¹, S(O)R¹¹, COR¹³—R¹⁸, COCHR¹⁸N(NO)R¹¹, COCHR¹⁸NR¹¹R¹² and COCHR¹⁸N⁺R¹¹R^{11a}R^{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹³ is selected from the group consisting of hydrogen, NHR^{12a}, NR¹¹R¹², NR¹¹Sug, N⁺R¹¹R^{11a}R^{11b}, R¹⁵, NR¹¹C(R^{11a}R^{11b})COR¹⁵ and group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R₁₁R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R¹²;
R¹⁵ is selected from N(R¹¹)NR^{11a}R¹², N(R¹¹)OR^{11a}, NR¹¹C(R^{11a}R^{11b})COR¹³;
R¹⁶ is selected from a group of the formula R—R⁵ or CH(NH₂)CH₂OH;
R¹⁷ is selected from SO₃H, SiR¹¹R^{11a}R^{11b}, SiOR¹¹OR^{11a}OR^{11b}, PR¹¹R^{11a}, P(O)R¹¹R^{11a}, P⁺R¹¹R^{11a}R^{11b};
R¹⁸ is selected from hydrogen, R¹, alkyl, aryl, phenyl-rhamnose-p, phenyl-(rhamnose-galactose)-p, phenyl-(galactose-galactose)-p, phenyl-O-methylrhamnose-p, wherein each alkyl and aryl can be substituted with 1 or more R¹⁹ or Sug,
R¹⁹ is selected from hydrogen, halogen, SH, SR²⁰, OH, OR²⁰, COOH, COR²⁰, COOR²⁰NO₂, NH₂, N(R²⁰)₂NHC(NH₂)═NH, CH(NH₂)═NH, NHOH, NHNH₂, N₃, NO, CN, N═NR²⁰, N═NR₁₂, SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰.
R²⁰ is selected from hydrogen, halogen, SH, OH, COOH, NO₂, NH₂, NHC(NH₂)═NH, CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring;
   and to their use as antiviral compounds and for the manufacture of a medicament to treat or prevent viral infections.

According to a particular embodiment, the present invention relates to compounds according to the general formula IV, V and VI, pharmaceutically acceptable salts, tautomers, and isomers thereof, wherein: 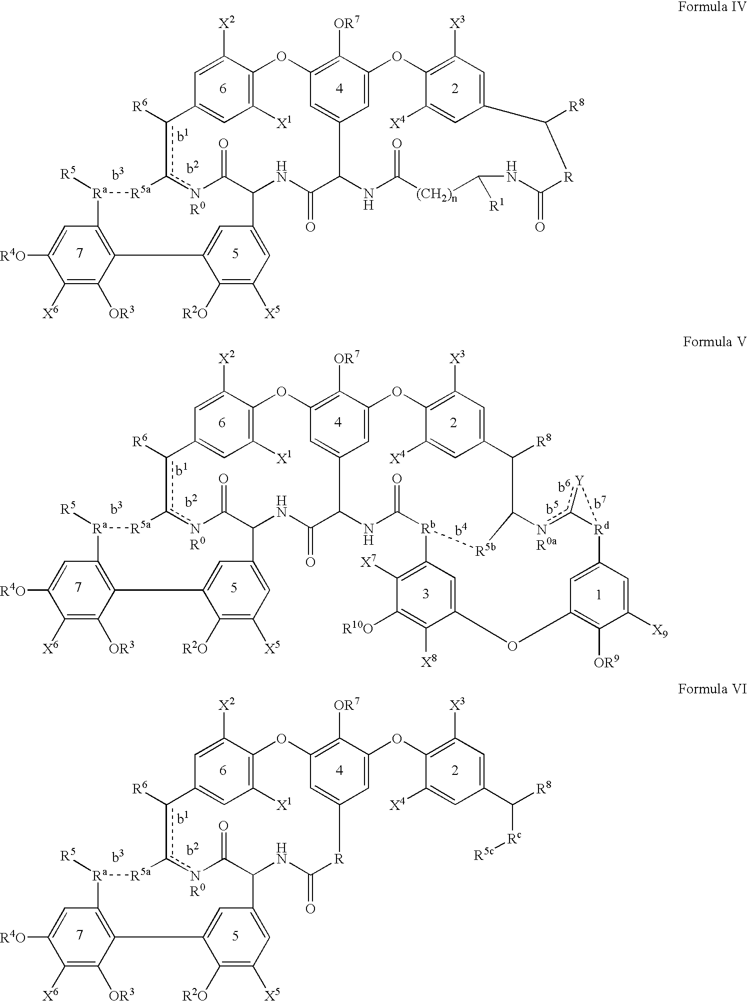
each b¹ and b² represent nihil, R⁶ represents R^{6a} and R⁰ represents hydrogen;
b³ represents an additional bond and R^{a}—R^{5a} represents CHNHCO;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R^{11a})CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and R^{d} represents R or a group of the formula (CH₂)_{q}CON(R¹¹)CH(CH₂OH)(CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R₁₁) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen and halogen;
X⁶ is CH₂R¹³;
X⁸ is selected from hydrogen and methyl;
R^{c} represents R and R^{5c} represents R⁵;
R is CHR¹³;
R¹ is selected from the group consisting of hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)-[O-phenyl(o-OR⁹, m-X⁹, m-R¹⁶)]-m, where t is 0, 1, 2, 3 or 4;
each R² and R⁴ are independently selected from hydrogen, R¹² and R¹⁷;
R³ is selected from hydrogen, R¹², R¹⁷, mannosyl and O-acetylmanosyl;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug and Sug is selected from glucosyl, ristosaminyl, N-acetylglucosaminyl, 4-epi-vancosaminyl, 3-epi-vancosaminyl, vancosaminyl, actinosaminyl, glucuronyl, 4-oxovancosaminyl, ureido-4-oxovancosaminyl and their derivatives;
R⁷ is selected from hydrogen, R¹², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug, wherein Sug is selected from glucosyl, mannosyl, ristosaminyl, N-acylglucosaminyl, N-acylglucuronyl, glucosaminyl, glucuronyl, 4-epi-vancosaminyl, 3-epi-vancosaminyl, vancosaminyl, actinosaminyl, acosaminyl, glucosyl-vancosaminyl, glucosyl-4-epi-vancosaminyl, glucosyl-3-epi-vancosaminyl, glucosyl-acosaminyl, glucosyl-ristosaminyl, glucosyl-actinosaminyl, glucosyl-rhamnosyl, glucosyl-olivosyl, glucosyl-mannosyl, glucosyl-4-oxovancosaminyl, glucosyl-ureido-4-oxovancosaminyl, glucosyl(rhamnosyl)-mannosyl-arabinosyl, glucosyl-2-O-Leu and their derivatives.

R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug, wherein Sug is selected from mannosyl, galactosyl and galactosyl-galactosyl;
R⁹ is selected from hydrogen, R¹², R¹⁷, galactosyl and galactosyl-galactosyl;
R¹⁰ is selected from hydrogen, R¹², R¹⁷, mannosyl or fucosyl;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹² is selected from the group consisting of hydrogen, acyl, amino-protecting group, carbamoyl, thiocarbamoyl, SO₂R¹¹, S(O)R¹¹, COR¹³—R¹⁸, COCHR¹⁸N(NO)R¹¹, COCHR¹⁸NR¹¹R¹² and COCHR¹⁸N⁺R¹¹R^{11a}R^{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R^{12a} is selected from the group consisting of hydrogen, COCHR¹⁸NR¹¹R¹², COCHR¹⁸N(NO)R¹¹, COCHR¹⁸N⁺R¹¹R^{11a}R^{11b} and COCHR¹⁸R¹³;
R¹³ is selected from the group consisting of hydrogen, NHR^{12a}, NR¹¹R¹², NR¹¹Sug, N⁺R¹¹R^{11a}R^{11b}, R¹⁵, NR¹¹C(R^{11a}R^{11b})COR¹⁵ and a group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R¹¹R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R¹²;
R¹⁵ is selected from N(R¹¹)NR₁₁ₐR¹², N(R¹¹)OR^{11a}, NR¹¹C(R^{11a}R^{11b})COR¹³;
R¹⁶ is selected from a group of the formula R—R⁵ or CH(NH₂)CH₂OH;
R¹⁷ is selected from SO₃H, SiR¹¹R^{11a}R^{11b}, SiOR¹¹OR^{11a}OR^{11b}, PR¹¹R^{11a}, P(O)R¹¹R^{11a}, P⁺R¹¹R^{11a}R^{11b};
R¹⁸ is selected from hydrogen, R¹, CH₃, CH₂CH(CH₃)₂, phenyl(p-OH, n-CI), phenyl-rhamnose-p, phenyl-(rhamnose-galactose)-p, phenyl-(galactose-galactose)-p, phenyl-O-methylrhamnose-p;
R¹⁹ is selected from hydrogen, halogen, SH, SR²⁰, OH, OR²⁰, COOH, COR²⁰, COOR²⁰NO₂, NH₂, N(R²⁰)₂NHC(NH₂)═NH, CH(NH₂)═NH, NHOH, NHNH₂, N₃, NO, CN, N═NR²⁰, N═NR¹², SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰.
R²⁰ is selected from hydrogen, halogen, SH, OH, COOH, NO₂, NH₂, NHC(NH₂)═NH, CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring;
   and to their use in the treatment of viral infections and to manufacture a medicament to treat or prevent viral infections.

In another particular embodiment, the invention relates to the use for the treatment or prevention of a viral infection or to the use to manufacture a medicament to treat or prevent a viral infection of derivatives of vancomycin, eremomycin, teicoplanin, ristomycin, cloroeremomycin, dechloroeremomycin, Des-(N-methyl-D-leucyl)-eremomycin aglycon, DA-40926, demannosyl-DA40926 or other structurally related glycopeptide antibiotics, including but not limted to their aglycon derivatives, their degradation derivatives and/or chemically modified derivatives.

More particularly, the present invention relates to compounds or glycopeptide antibiotics or derivatives thereof according to the general formula Z and/or I, II, III and/or IV, V and VI as defined above, provided that:
the compounds are not natural glycopeptide antibiotics, such as vancomycin, eremomycin, teicoplanin;
the compounds are not compounds with the codes 1 to 55 as in example 1 of this application;
the compounds are not compounds with the codes 1 to 172 as in example 1 of this application;
the compound is not a compound selected out of the compounds as exemplified in exemple 1 of this application.

In a particular embodiment, the present invention relates to glycopeptide antibiotics and derivatives thereof according to the general formula Z and/or I, II, III and/or IV, V and VI as defined above, with the exclusion of a selection of compounds selected from any of the compounds exemplified in example 1.

In yet another particular embodiment, the present invention relates to the use of glycopeptide antibiotic and derivatives thereof selected from the group consisting of the compounds with the code 40, 88, 98, 115, 132, 145 or 146 of example 1 of this application, for the preparation of a medicament for the treatment or prevention of a viral infection, wherein said viral infection is an infection of Herpes Simplex virus. In another particular embodiment, the present invention relates to the use of glycopeptide antibiotic and derivatives thereof selected from the group consisting of the compounds with the code 6, 7, 8, 16, 17, 18, 20, 21, 24, 25, 27, 28, 31, 32, 33, 35, 36, 37, 39, 40, 41, 46, 59, 68, 76, 77, 81, 89, 90, 98, 113, 115, 117, 119, 120, 121, 122, 123, 124, 125, 126, 127, 128, 132, 136, 137, 140, 141, 142, 143, 145, 146 and 169 of example 1 of this application, for the preparation of a medicament for the treatment or prevention of a viral infection, wherein said viral infection is an infection of Varizaella Zoster virus. In still another particular embodiment, the present invention relates to the use of glycopeptide antibiotic and derivatives thereof selected from the group consisting of the compounds with the code 18, 21, 25, 26, 27, 31, 37, 39, 59, 68, 89, 112, 122, 124, 125, 127 of example 1 of this application, and 146, for the preparation of a medicament for the treatment or prevention of a viral infection, wherein said viral infection is an infection of Cytomegalovirus. Another particular embodiment of the present invention relates to the use of glycopeptide antibiotic and derivatives thereof selected from the group consisting of the compounds with the code 86, 87 and 126 of example 1 of this application, for the preparation of a medicament for the treatment or prevention of a viral infection, wherein said viral infection is an infection of Hepatitis C virus or BVDV. In yet another particular embodiment, the present invention relates to the use of glycopeptide antibiotic and derivatives thereof selected from the group consisting of the compounds with the code 1, 5, 7, 9, 13, 19, 28, 30, 31, 41, 47, 51, 52, 53, 54, 55, 63, 64, 99, 100, 101, 102, 106, 107, 108, 109, 124, 125, 159, 160, 161, 162, 163, 165, 166, 167, 170 and 53 of example 1 of this application, for the preparation of a medicament for the treatment or prevention of a viral infection, wherein said viral infection is an infection of FCV or SARS causing virus.

The present invention further relates to the use of glycopeptide antibiotics and their derivatives, more in particular of a compound of the general formula Z or the formula I, II and III, optionally of the formula IV, V and VI as a medicine, to the use of such compounds in the treatment of a viral infection or to manufacture a medicament to treat or prevent viral infections in a subject. The invention also relates to the use of glycopeptide antibiotics and their derivatives, more particularly of a compound of formula Z or I, II and III, optionally of the formula IV, V and VI as a pharmaceutically active ingredient, especially as an inhibitor of the viral replication, more preferably as an inhibitor of the replication of a virus of the family of the Flaviviridae, the retroviridae (i.e. Lentivirinae), the herpes viridae and the Coronaviridae, and yet more preferably as an inhibitor of the replication of BVDV, HCV, HIV, HSV, CMV, VZV, FCV and of the virus causing SARS. Therefore, the invention also relates to the use of glycopeptide antibiotics and their derivatives, more particularly of a compound of formula Z or I, II and III, optionally of the formula IV, V and VI for the manufacture of a medicine or a pharmaceutical composition having antiviral activity for the prevention and/or treatment of viral infections in humans and mammals. The present invention further relates to a method of treatment of a viral infection in a mammal, including a human, comprising administering to the mammal in need of such treatment a therapeutically effective amount of a glycopeptide antibiotic and their derivatives, more particularly of a compound of formula Z or I, II and III, more particularly of the formula IV, V and VI as an active ingredient, optionally in a mixture with at least a pharmaceutically acceptable carrier.

In yet another embodiment, the present invention relates to the use of glycopeptide antibiotic derivatives for the preparation of a medicament for the treatment or prevention of a viral infection, optionally excluding the natural glycopeptide antibiotics.

According to a particular embodiment, the present invention relates to compounds selected from the group of compounds 56 to 172 of example 1 of this application, the pharmaceutically acceptable salts, tautomers, and isomers thereof. In another particular embodiment, the present invention relates to the the use of compounds selected from the group of compounds 1 to 172 of example 1 of this application, the pharmaceutically acceptable salts, tautomers, and isomers thereof, for the treatment of viral infections or for the manufacture of a medicament to treat or prevent viral infections.

The invention also relates to methods for the preparation of glycopeptide antibiotic derivatives, more particularly of compounds of formula Z or I, II and III, more particularly of the formula IV, V and VI, more particularly to methods for the preparation of the compounds specifically disclosed herein, to pharmaceutical compositions comprising them in a mixture with at least a pharmaceutically acceptable carrier, the active ingredient optionally being in a concentration range of about 0.1-100% by weight, and to the use of these derivatives namely as antiviral drugs, more particularly as drugs useful for the treatment of subjects suffering from HIV, HCV, BVDV, HSV, VZV, CMV, FCV infections or of virally caused SARS.

The present invention also relates to methods of structurally modifying said compounds for increasing the antiviral activity and methods of structurally modifying said compounds for decreasing or removing antibacterial activity while maintaining antiviral activity. The present invention further relates to the selection of optimal antiviral glycopeptide derivatives, namely by following the steps of synthesising new glycopeptide derivatives, screening in a random order for antibacterial activity, and testing the cellular toxicity of the derivatives by methods known in the art and followed by selecting the derivatives with low or no antibacterial and toxic effect and high antiviral activity.

### DETAILED DESCRIPTION OF THE INVENTION

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

As used herein and unless otherwise stated, the term “halogen” means any atom selected from the group consisting of fluorine (F), chlorine (Cl), bromine (Br) and iodine (I).

The term “alkyl” refers to straight or branched (normal, secondary, tertiary) C₁-C₂₄ hydrocarbon chains without or with 1 or more heteroatoms in the hydrocarbon chain. The number and position of heteroatoms is variable. Each heteroatom can independently be selected from O, N, S, SO, SO₂, P or B. Examples are methyl, ethyl, 1-propyl, 2-propyl, 1-butyl, 2-methyl-1-propyl(i-Bu), 2-butyl(s-Bu)2-methyl-2-propyl(t-Bu), 1-pentyl(n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl.

The term “alkylene” as used herein each refer to a saturated, branched or straight chain hydrocarbon radical of 1-24 carbon atoms, and having two monovalent radical centers derived by the removal of two hydrogen atoms from the same or two different carbon atoms of a parent alkane, without or with 1 or more heteroatoms in the hydrocarbon chain. Typical alkylene radicals include, but are not limited to: methylene (—CH2—) 1,2-ethyl(—CH2CH2—), 1,3-propyl(—CH2CH2CH2—), 1,4-butyl(—CH2CH2CH2CH2—), and the like.

As used herein and unless otherwise stated, the term “cycloalkyl” means a C₃-C₂₄ monocyclic or polycyclic saturated hydrocarbon chain monovalent radical having from 3 to 24 carbon atoms, such as for instance cyclopropyl, cyclobutyl, cyclopentyl, cyclohexyl, cycloheptyl, cyclooctyl, cyclododecyl, bicyclopentyl, bicyclohexyl, bicycloheptyl, bornyl, norbornyl, fenchyl, trimethyltricycloheptyl or adamantyl and the like.

The term “alkenyl” as used herein is C₂-C₂₄ normal, secondary or tertiary hydrocarbon chain with at least one site of unsaturation, i.e. a carbon-carbon, sp2 double bond and without or with 1 or more heteroatoms in the hydrocarbon chain. Each heteroatom can independently be selected from O, N, S, SO, SO₂, P or B. The term “cycloalkenyl” as used herein is a C₃-C₂₄ mono- or polycyclic hydrocarbon chain with at least one site of unsaturation, i.e. a carbon-carbon, sp2 double bond. Examples include, but are not limited to: ethylene or vinyl(—CH═CH2), allyl(—CH2CH═CH2), cyclopentenyl(—C₅H₇), cyclohexenyl(—C₆H₉), 2-methyl-cyclohexenyl, and 5-hexenyl(—CH2 CH2CH2CH2CH═CH2). The double bond may be in the cis or trans configuration.

The term “alkynyl” as used herein refers to C₂-C₂₄ normal, secondary or tertiary hydrocarbon chain with at least one site of unsaturation, i.e. a carbon-carbon, sp triple bond and without or with 1 or more heteroatoms in the hydrocarbon chain. Each heteroatom can independently be selected from O, N, S, SO, SO₂, P or B. The term “cycloalkynyl” as used herein is a C₃-C₂₄ mono- or polycyclic hydrocarbon chain with at least one site of unsaturation, i.e. a carbon-carbon, sp triple bond. Examples include, but are not limited to: acetylenic(—C°CH) and propargyl(—CH2C°CH). (note: ° means a triple bond)

The term “heterocyclic ring”, as used herein, refers to saturated or unsaturated, monocyclic, bicyclic, tricyclic and other polycyclic C₃-C₂₄ hydrocarbon chains (cycloalkyl, cycloalkenyl, cycloalkynyl) with 1 or more heteroatoms selected from S, O, N or B. Examples of heterocyclic rings are piperazinyl, piperidinyl, morpholinyl, quinuclidinyl, borabicyclononyl, crown ethers, azacrowns, thiacrowns, and the like.

The term “aryl” as used herein refers to an aromatic hydrocarbon radical of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, spiro, anthracene, biphenyl, and the like. Therefore the term includes aromatic C₆ membered organic monocyclic ring, aromatic C₉-C₁₀ membered organic fused bicyclic rings, aromatic C₁₂-C₁₄ membered organic fused tricyclic rings and aromatic C₁₄-C₁₆ membered organic fused tetracyclic rings. Examples are phenyl, biphenyl, triphenyl, naphtyl, fluorenyl, phenanthrenyl and the like.

“Arylalkyl” as used herein refers to an alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

“Heteroaryl” refers to aryl with 1 or more heteroatoms in the aromatic hydrocarbon ring system. The heteroatoms can be selected from O, N and S. The nitrogen and sulfur atoms of these rings are optionally oxidized, and the nitrogen heteroatoms are optionally quarternized. Examples are pyridyl, dihydropyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl, indolyl, quinolyl, piperonyl, oxafluorenyl, benzothienyl and the like.

By way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

As described previously, alkyl, alkylene, alkenyl, alkynyl, cycloalkyl, cycloalkenyl, cycloalkynyl, aryl, heteroaryl, and arylalkyl groups and heterocyclic rings can also be substituted in the invention. Typically, they are substituted with one or more R¹⁹.

The term “acyl”, as used herein, refers to a group of the formula: —COR¹¹, —COOR¹¹ or —CSR¹¹ wherein R¹¹ is described above.

The term “carbamoyl”, as used herein, refers to a group of the formula: —CONR¹¹R^{11a} or —CONHR¹² wherein R¹¹, R^{11a} and R¹² are described above.

The term “thiocarbamoyl” refers to group of the formula: —CSNHR¹² or —C⁺(SR¹¹)NHR¹², wherein R¹¹ and R¹² are described above.

The term “amino-protecting group” refers to those groups known in the art to be suitable for protecting the amino group during the acylation reaction. Such groups are well recognized, and selecting a suitable group for this purpose will be apparent. The tert-butoxycarbonyl (Boc), adamantyloxycarbonyl (Adoc), fluorenylmethoxycarbonyl (Fmoc) and carbobenzoxycarbonyl (Cbz) groups are examples of suitable amino-protecting groups.

The term “carbohydrate” or “Sugar” (“Sug”) refers to any cyclic or acyclic carbohydrate or multiple carbohydrates coupled to each other. Examples of carbohydrates are glucosyl, mannosyl, ristosaminyl, N-acylglucosaminyl, N-acylglucuronyl, glucosaminyl, glucuronyl, 4-epi-vancosaminyl, 3-epi-vancosaminyl, vancosaminyl, actinosaminyl, acosaminyl, glucosyl-vancosaminyl, glucosyl-4-epi-vancosaminyl, glucosyl-3-epi-vancosaminyl, glucosyl-acosaminyl, glucosyl-ristosaminyl, glucosyl-actinosaminyl, glucosyl-rhamnosyl, glucosyl-olivosyl, glucosyl-mannosyl, glucosyl-4-oxovancosaminyl, glucosyl-ureido-4-oxovancosaminyl, glucosyl(rhamnosyl)-mannosyl-arabinosyl, glucosyl-2-O-Leu. The carbohydrates can also be derivatised and these terms also refer to derivatives of carbohydrates. Derivatives of carbohydrates comprise carbohydrates substituted with chemical groups containing heteroatoms (O, N, S), such as amino, carboxy, hydroxy and oxo groups. Typical carbohydrate derivatives comprising carbohydrates substituted with NR¹¹R¹², N⁺R¹¹R^{11a}R^{11b}, COR¹¹, COR¹³, COR¹⁵, O—R¹², O—R¹⁷, C═NOR¹¹, CHNHOR¹¹, C═NNR¹¹R¹² or C═NNHCONR¹¹R¹².

Any substituent designation that is found in more than one site in a compound of this invention shall be independently selected.

The term “glycopeptide antibiotics” refers to the natural glycopeptide antibiotics (glycopeptidic molecules produced by microorganisms such as actinomycetes with antibacterial activity). They are mostly compounds of relatively high molecular weight and structurally, they comprise a polypeptide core aglycone structure having phenolic amino acids and one or more peripheral carbohydrate moieties. Examples are vancomycin, eremomycin, chloreremomycin, teicoplanin, DA-40926, Demannosyl-DA40926, ristocetin, A35512, avoparcin, actaplanin, AAD-216, A477, OA7633, AM 374, actinoidin, ristomycin and the like.

“Glycopeptide antibiotic derivatives” comprise natural, semisynthetic or synthetic derivatives, partially degraded (aglycon derivatives) or modified with chemical or enzymatic procedures in the peptide or sugar moieties, the glycopeptide antibiotic aglycons and also products of their partial degradation with the peptide core destroyed or modified in peptide core and in sugar moieties.

Any substituent designation that is found in more than one site in a compound of this invention shall be independently selected.

As used herein and unless otherwise stated, the term “amino-acid” refers to a radical derived from a molecule having the chemical formula H₂N—CHR²³R²²—COOH, wherein R²³R²² is the side group of atoms characterizing the amino-acid type; said molecule may be one of the 20 naturally-occurring amino-acids or any non naturally-occurring amino-acid. Esters of amino acids included within this definition are substituted at one or more caboxyl groups with C₁₋₆ alkyl. This is the case even when the amino acid is bonded through carboxyl because some amino acids contain more than one caboxyl groups, and in this case the unbonded carboxyl optionally is esterified. R²²—R²³ is C₁-C₆ alkyl or C₁-C₆ alkyl substituted with amino, carboxyl, amide, carboxyl (as well as esters, as noted above), hydroxyl, C₆-C₇ aryl, guanidinyl, imidazolyl, indolyl, sulfhydryl, sulfoxide, and/or alkylphosphate. R²² R²³ also is taken together with the amino acid anitrogen to form a proline residue (R²² R²³ is —(CH₂)₃—). However, R²²—R²³ is generally the side group of a naturally-occurring amino acid such as H, —CH₃, —CH(CH₃)₂, —CH₂—CH(CH₃)₂, —CHCH₃—CH₂—CH₃, —CH₂—C₆H₅, —CH₂CH₂—S—CH₃, —CH₂OH, CH(OH)—CH₃, —CH₂SH, —CH₂—C₆H₄OH, —CH₂—CO—NH₂, —CH₂—CH₂—CO—NH₂, —CH₂—COOH, —CH₂—CH₂—COOH, —(CH₂)₄—NH₂ and —(CH₂)₃—NH—C(NH₂)—NH₂. R²²R²³ also includes 1-guanidinoprop-3-yl, benzyl, 4-hydroxybenzyl, imidazol-4-yl, indol-3-yl, methoxyphenyl and ethoxyphenyl.

Optionally the amino acid residue is a hydrophobic residue such as mono- or di-alkyl or aryl amino acids, cycloalkylamino acids and the like. Optionally, the residue does not contain a sulfhydryl or guanidino substituent. Optionally, the amino acid is a phenolic amino acid.

Naturally-occurring amino acid residues are those residues found naturally in plants, animals or microbes, especially proteins thereof. Polypeptides most typically will be substantially composed of such naturally-occurring amino acid residues. These amino acids are glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine aud hydroxyproline. Additionally, unnatural amino acids, for example, valanine, phenylglycine and homoarginine are also included.

Substituents optionally are designated with or without bonds. Regardless of bond indications, if a substituent is polyvalent (based on its position in the structure referred to), then any and all possible orientations of the substituent are intended.

The formula's Z, A, I, II, III, IV, V and VI depict optional single or double bonds. It will be understood that bonds are present such that this is electronically possible. These formulas are intended to embrace all possible tautomers.

The compounds of the invention optionally are bound covalently to an insoluble matrix and used for affinity chromatography (separations, depending on the nature of the groups of the compounds, for example compounds with many free hydroxyl functions are useful in hydrophylic affinity separations.

The present invention includes a class of natural glycopeptide antibiotics and their derivatives and a class of compounds with structural similarity to said natural glycopeptide antibiotics which possess antiviral activity such as the anti-retroviral activity, anti-flaviviral, anti-herpes and anti-coronaviral activity of presented examples. Such compounds can be natural glycopeptide antibiotics, with structures as for instance disclosed in K. C. Nicolaou, C.N.C. et al. Chem. Int. Ed., 1999, V. 38, p. 2096-2152 and B. Cavalleri & F. Parenti. Encyclopedia of Chemical Technology, 1992, V. 2, p. 995-1018. The invention also includes derivatives of glycopeptide antibiotics, which have been structurally engineered or modified to decrease or remove completely or partially the antibacterial activity while still comprising antiviral activity. Several compounds of the invention were tested for their antibacterial activity and showed to be not or less active as anti-bacterial than the parent compound. Antibacterial assays that can be used for this purpose are well known in the art. The present invention also provides synthetic, semisynthetic or biosynthetic derivatives of natural glycopeptide antibiotics of the general formula Z, or I, II, III or IV, V and VI. The above mentioned compounds may be engineered to be less active or inactive antibacterials at therapeutically effective antiviral doses and it also has been demonstrated by this invention that they can be engineered to have no mammalian cell toxicity at therapeutically effective antiviral doses. The compounds are selected for antiviral activity and low mammalian cell toxicity and eventually may be selected as additional property antibacterial inactivity in antiviral activity assays such as the anti-HIV assays of present invention, a cytostatic activity assay of the state of the art or the cytostatic activity assay on the mammalian cell lines (L1210, Molt4/C8 or CEM) of present invention and additional antibacterial assays of the state of the art.

The compounds of the invention are employed for the treatment or prophylaxis of viral infections, more particularly flaviviral, retroviral, herpes or coronaviral infections, in particular, HCV, BVDV, HIV, HSV, CMV, YFV, FCV, VZV and SARS virus. When using one or more glycopeptide antibiotics or their derivatives, or more particularly derivatives of the formula Z or I, II and III as defined herein:
the active ingredients of the compound(s) may be administered to the mammal (including a human) to be treated by any means well known in the art, i.e. orally, intranasally, subcutaneously, intramuscularly, intradermally, intravenously, intra-arterially, parenterally or by catheterization.
the therapeutically effective amount of the preparation of the compound(s), especially for the treatment of viral infections in humans and other mammals, preferably is a flaviviral, retroviral, herpes or coronaviral enzyme inhibiting amount. More preferably, it is a flaviviral, retroviral, herpes or coronaviral replication inhibiting amount or a flaviviral, retroviral, herpes or coronaviral enzyme inhibiting amount of the derivative(s) of formula Z or I, II and III as defined herein corresponds to an amount which ensures a plasma level of between 1 μg/ml and 100 mg/ml, optionally of 10 mg/ml. This can be achieved by administration of a dosage of in the range of 0.001 mg to 20 mg, preferably 0.01 mg to 5 mg, preferably 0.1 mg to 1 mg per day per kg bodyweight for humans. Depending upon the pathologic condition to be treated and the patient's condition, the said effective amount may be divided into several sub-units per day or may be administered at more than one day intervals.

The present invention further relates to a method for preventing or treating a viral infections in a subject or patient by administering to the patient in need thereof a therapeutically effective amount of glycopeptide antibiotics and their derivatives of the present invention. The therapeutically effective amount of the preparation of the compound(s), especially for the treatment of viral infections in humans and other mammals, preferably is a flaviviral, retroviral, herpes or coronaviral enzyme inhibiting amount. More preferably, it is a flaviviral, retroviral, herpes or coronaviral replication inhibiting amount or a flaviviral, retroviral, herpes or coronaviral enzyme inhibiting amount of the glycopeptide antibiotics and their derivatives, more particularly of the derivative(s) of formula Z or I, II and III as defined herein. Suitable dosage is usually in the range of 0.001 mg to 20 mg, preferably 0.01 mg to 5 mg, preferably 0.1 mg to 1 mg per day per kg bodyweight for humans. Depending upon the pathologic condition to be treated and the patient's condition, the said effective amount may be divided into several sub-units per day or may be administered at more than one day intervals.

As is conventional in the art, the evaluation of a synergistic effect in a drug combination may be made by analyzing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

$${CI}_{x} = \frac{{ED}_{x}^{1 c}}{{ED}_{x}^{1 a}} + \frac{{ED}_{x}^{2 c}}{{ED}_{x}^{2 a}}$$

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively.

Synergistic activity of the pharmaceutical compositions or combined preparations of this invention against viral infection may also be readily determined by means of one or more tests such as, but not limited to, the isobologram method, as previously described by Elion et al. in J. Biol. Chem. (1954) 208:477-488 and by Baba et al. in Antimicrob. Agents Chemother. (1984) 25:515-517, using EC₅₀ for calculating the fractional inhibitory concentration (hereinafter referred as FIC). When the minimum FIC index corresponding to the FIC of combined compounds (e.g., FICₓ+FIC_{y}) is equal to 1.0, the combination is said to be additive; when it is beween 1.0 and 0.5, the combination is defined as subsynergistic, and when it is lower than 0.5, the combination is by defined as synergistic. When the minimum FIC index is between 1.0 and 2.0, the combination is defined as subantagonistic and, when it is higher than 2.0, the combination is defined as antagonistic.

This principle may be applied to a combination of different antiviral drugs of the invention or to a combination of the antiviral drugs of the invention with other drugs that exhibit anti-retroviral, anti-flaviviral, anti-herpes or anti-coronaviral activity.

The invention thus relates to a pharmaceutical composition or combined preparation having synergistic effects against a viral infection and containing:

Either:

A)
(a) a combination of two or more of the glycopeptide antibiotics, their derivatives or more particularly compounds according to formula Z or I, II and III of the present invention, and
(b) optionally one or more pharmaceutical excipients or pharmaceutically acceptable carriers, for simultaneous, separate or sequential use in the treatment or prevention of a viral infection or
B)
(c) one or more anti-viral agents, and
(d) at least one of the glycopeptide antibiotics, their derivatives or more particularly compounds according to formula Z or I, II and III of the present invention, and
(e) optionally one or more pharmaceutical excipients or pharmaceutically acceptable carriers, for simultaneous, separate or sequential use in the treatment or prevention of a viral infection.

Suitable anti-viral agents for inclusion into the synergistic antiviral compositions or combined preparations of this invention include, for instance, interferon-alfa (either pegylated or not), nucleoside reverse transcriptase (RT) inhibitors (i.e. zidovudine, didanosine, stavudine, lamivudine, zalcitabine and abacavir), non-nucleoside reverse transcriptase inhibitors (i.e. nevirapine, delavirdine and efavirenz), protease inhibitors (i.e. saquinavir, indinavir, ritonavir, nelfinavir, amprenavir and lopinavir), fusion inhibitor enfuvirtide, ribavirin, vidarabine, acyclovir, gancyclovir, amantadine, rimantadine and other selective inhibitors of the replication of BVDV, HCV, HIV, HSV, VZV, CMV, FCV and SARS virus.

The pharmaceutical composition or combined preparation with synergistic activity against viral infection according to this invention may contain glycopeptide antibiotics, their derivatives or more particularly compounds according to formula Z or I, II and III of the present invention over a broad content range depending on the contemplated use and the expected effect of the preparation. Generally, the content of the glycopeptide antibiotics, their derivatives or more particularly compounds according to formula Z or I, II and III of the present invention of the combined preparation is within the range of 0.1 to 99.9% by weight, preferably from 1 to 99% by weight, more preferably from 5 to 95% by weight.

According to a particular embodiment of the invention, the compounds of the invention may be employed in combination with other therapeutic agents for the treatment or prophylaxis of flaviviral, retroviral, herpes or coronaviral infections, such as for example also corticosteroids in the case of SARS. The invention therefore relates to the use of a composition comprising:
(a) one or more glycopeptide antibiotics, their derivatives or more particularly compounds according to formula Z or I, II and III of the present invention, and
(b) one or more flaviviral, retroviral, herpes or coronaviral enzyme inhibitors as biologically active agents in respective proportions such as to provide a synergistic effect against a viral infection, particularly a flaviviral, retroviral, herpes or coronaviral infection in a mammal, for instance in the form of a combined preparation for simultaneous, separate or sequential use in viral infection therapy, such as of HCV, BVDV, HIV, HSV, VZV, YFV, FCV, CMV and SARS virus. Examples of such further therapeutic agents for use in combinations include agents that are effective for the treatment or prophylaxis of these infections, including interferon alpha, ribavirin, and other mentioned before. More examples are compounds falling under the scope of patents or patent applications handling with inhibitors of viral infections, more particularly flaviviral, retroviral, herpes and coronaviral infections. For example, compounds falling within the scope of disclosure EP1162196, WO 03/010141, WO 03/007945 and WO 03010140, a compound falling within the scope of disclosure WO 00/204425, and other patents or patent applications within their patent families or all the foregoing filings and/or an inhibitor of flaviviral protease and/or one or more additional flavivirus polymerase inhibitors, can be used.
   When using a combined preparation of (a) and (b):
   the active ingredients (a) and (b) may be administered to the mammal (including a human) to be treated by any means well known in the art, i.e. orally, intranasally, subcutaneously, intramuscularly, intradermally, intravenously, intra-arterially, parenterally or by catheterization.
   the therapeutically effective amount of the combined preparation of (a) and (b), especially for the treatment of viral infections in humans and other mammals, particularly is a flaviviral, retroviral, herpes or coronaviral enzyme inhibiting amount. More particularly, it is a flaviviral, retroviral, herpes or coronaviral replication inhibiting amount of derivative (a) and a flaviviral, retroviral, herpes or coronaviral enzyme inhibiting amount of inhibitor (b). Still more particularly when the said flaviviral, retroviral, herpes or coronaviral enzyme inhibitor (b) is a polymerase inhibitor, its effective amount is a polymerase inhibiting amount. When the said flaviviral or picornaviral enzyme inhibitor (b) is a protease inhibitor, its effective amount is a protease inhibiting amount.
   ingredients (a) and (b) may be administered simultaneously but it is also beneficial to administer them separately or sequentially, for instance within a relatively short period of time (e.g. within about 24 hours) in order to achieve their functional diffusion in the body to be treated.

The invention also relates to the glycopeptide antibiotics and their derivatives, more particularly compounds of formula Z or I, II and III of this invention being used for inhibition of the replication of other viruses than BVDV, HCV, HIV, YFV, HSV, CMV, VZV, FCV or SARS virus, particularly for the inhibition of other flaviviruses, herpes viruses, retroviruses or coronaviruses or picornaviruses, with in particular Dengue virus, hepatitis B virus, hepatitis G virus, Classical Swine Fever virus or the Border Disease Virus, epstein bar virus and also for other viral families such as the Picornaviruses (i.e. enterovirus, rhinovirus, Coxsackie virus), orthomyxoviridae (i.e. influenza), paramyxoviridae (i.e. parainfluenza, human metapneumavirus, respiratory syncytial virus (RSV)), rhabdoviridae (i.e. rabies), bunyaviridae (i.e. hantavirus), filoviridae (i.e. marburg, ebola), Poxyiridae (i.e. variola), Adenoviridae, Papovaviridae (i.e. human papilloma virus) and others.

The present invention further provides veterinary compositions comprising at least one active ingredient as above defined together with a veterinary carrier therefor, for example in the treatment of BVDV or FCV. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

More generally, the invention relates to glycopeptide antibiotics and their derivatives, more particularly compounds of formula Z or I, II and III of this invention being useful as agents having biological activity (particularly antiviral activity) or as diagnostic agents. Any of the uses mentioned with respect to the present invention may be restricted to a non-medical use, a non-therapeutic use, a non-diagnostic use, or exclusively an in vitro use, or a use related to cells remote from an animal.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state, any and all protonated forms of the compounds are intended to fall within the scope of the invention.

The term “pharmaceutically acceptable salts” as used herein means the therapeutically active non-toxic salt forms which the glycopeptide antibiotics and their derivatives, more particularly compounds of formula Z or I, II and III of this invention are able to form. Therefore, the compounds of this invention optionally comprise salts of the compounds herein, especially pharmaceutically acceptable non-toxic salts containing, for example, Na+, Li+, K+, Ca+2 and Mg+2. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li+, Na+, and K+. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which glycopeptide antibiotics and their derivatives, more particularly compounds of formula Z or I, II and III of this invention as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX4+ (wherein X is C1-C4 alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na+ and NX4+ (wherein X typically is independently selected from H or a C1-C4 alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

Each compound of the present invention may be a pure stereoisomer coupled at each of its chiral centers or it may be inverted at one or more of its chiral centers. It may be a single stereoisomer or a mixture of two or more stereoisomers. If it is a mixture, the ratio may or may not be equimolar. In a particular embodiment, the compound is a single stereoisomer and in a more particular embodiment, the stereochemistry of the peptide core of the compounds of the invention containing six amino acids (2-7) is 2(R), 3(S), 4(R), 5(R), 6(S) and 7(S).

The term “isomers” as used herein means all possible isomeric forms, including tautomeric and sterochemical forms, which glycopeptide antibiotics and their derivatives, more particularly compounds of formula Z or I, II and III of this invention may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the glycopeptide antibiotics and their derivatives, more particularly compounds of formula Z or I, II and III of this invention may have at least one chiral center) of the basic molecular structure, as wel as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantionierically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine(amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCeI™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of formula Z or I, II and III may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by inicronisation, for instance in view to obtain them in the form of microspheres usually having a diameter of about 1 to 10 gm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw’, 2 d ed. (Hanser Verlag, Vienna, 1981) and “Encyclopaedia of Surfactants, (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other. therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

A specific formulation for glycopeptide antibiotics is the combination with cyclodextrin as described in WO01/82971

Preferred unit dosage formulations are those containing a daily dose or unit daily sub-dose, as herein above recited, or an appropriate fraction thereof, of an active ingredient.

It should be understood that in addition to the ingredients particularly mentioned above, the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

### EXAMPLES

The following examples illustrate the present invention without being limited thereto. Examples are given of compounds, of methods and materials for the preparation of the compounds and also pharmacological examples are shown.

In the examples and tables regularly used abbreviations and terms are:
EC₅₀: 50% effective concentration, or compound concentration required to inhibit virus-induced cytopathicity by 50%
MCC, minimal cytotoxic concentration, or compound concentration required to cause a microscopically visible morphological change of the cell culture
CC₅₀, 50% cytostatic/cytotoxic concentration or compound concentration required to inhibit HEL cell proliferation by 50% or to reduce MDBK, Vero or FCK cell viability by 50%.
MTC, or minimal toxic concentration, or compound concentration required to afford a ≧20% reduction of the metabolic activity of uninfected cells by means of the MTS method.
Abbreviations used: HSV-1, herpes simplex virus type 1; HSV-2, herpes simplex virus type 2; VZV, varicella-zoster virus, CMV, human cytomegalovirus; BVDV, bovine viral diarrhea virus; YFV, Yellow Fever virus; FCV, feline corona virus; SARS, human corona (SARS, strain Frankfurt-1) virus; HEL, human embryonic lung fibroblasts; MDBK, Madin-Darby bovine kidney cells, Vero, simian kidney cells; FCK, feline Crandel kidney cells.
Blanco fields in tables showing anti-viral activity mean that the compounds have not been tested.

### Example 1

### Tables 1 to 8 Represent the Structures of Prepared Compounds as Examples and Their Respective Codes

In this application several compounds of the invention are referred to with a code as specified hereunder.

TABLE 1
Vancomycin type glycopeptides and their derivatives

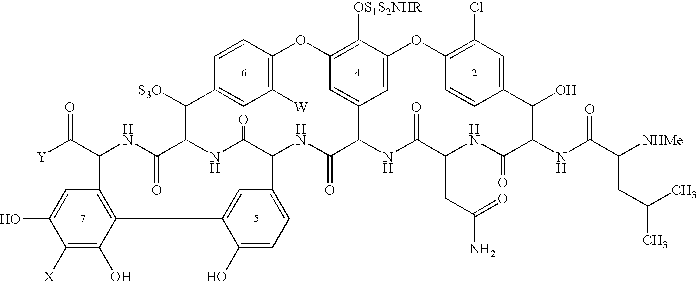

| | | | | | | |
|---|---|---|---|---|---|---|
| | | | | | | MW |
| | | | | | MW | [M + 1H] |
| Code no. | X | Y | R | Brutto formula | Calc. | found |

Vancomycin (Van) and its derivatives W = CI, S₁ = Glc, S₂ = vancosamine, S₃ = H

| | | | | | | |
|---|---|---|---|---|---|---|
| Van | H | OH | H | C₆₆H₇₅N₉O₂₄Cl₂ | 1448 | 1449 |
| 56 | H | NHC₁₀H₂₁ | H | C₇₆H₉₆N₁₀O₂₃Cl₂ | 1587 | 1588 |
| 57 | H | NHBnPhCI-p | H | C₇₉H₈₅N₁₀O₂₃Cl₃ | 1647 | 1648 |
| 2 | H | NH(CH₂)₃N⁺Me2 | H | C₈₁H₁₀₈N₁₁O₂₃Cl₂ | 1673 | 1674 |
| 1 | CH₂N[CH₂CH₂]₂ | OH | H | C₈₂H₉₉N₁₁O₂₄Cl₂ | 1694 | 1695 |
| | NBnBu-p | | | | | |
| 58 | H | OH | COCH₂NHB | C₈₁H₈₇N₁₀O₂₅Cl | 1705 | 1706 |
| | | | nPhCl-p | | | |
| 59 | H | OH | BnPhCI-p | C₇₉H₈₄N₉O₂₄Cl₃ | 1648 | 1649 |

Eremomycin (Ere) and its derivatives W = H, S₁ = Glc,
S₂ = S₃ = eremosamine

| | | | | | | |
|---|---|---|---|---|---|---|
| Ere | H | OH | H | C₇₃H₈₉N₁₀O₂₆Cl | 1556 | 1557 |
| 60 | H | NHMe | H | C₇₄H₉₃N₁₁O₂₅Cl | 1570 | 1571 |
| 61 | CH₂NHC₁₀H₂₁ | OH | H | C₈₄H₁₁₂N₁₁O₂₆Cl | 1725 | 1726 |
| 62 | CH₂NMecH₂ | OH | H | C₈₁H₁₀₆N₁₁O₃₁Cl | 1764 | 1765 |
| | (CHOH)₄CH₂OH | | | | | |
| 63 | CH₂NHC₁₈H₃₇ | OH | H | C₉₂H₁₂₈N₁₁O₂₆Cl | 1837 | 1838 |
| 64 | CH₂NHC₁₂H₂₅ | OH | H | C₈₆H₁₁₆N₁₁O₂₆Cl | 1753 | 1754 |
| 65 | H | NHC₁₀H₂₁ | H | C₈₃H₁₁₀N₁₁O₂₃Cl | 1693 | 1694 |
| 66 | H | NHBnCI-p | H | C₈₀H₉₅N₁₁O₂₅Cl₂ | 1681 | 1682 |
| 67 | CH₂NHBnPh-p | OH | H | C₈₇H₁₀₂N₁₁O₂₆Cl | 1751 | 1752 |
| 68 | H | OH | C_{10H}21 | C₈₃H₁₀₉N₁₀O₂₆Cl | 1696 | 1697 |
| 69 | H | OH | BnPh-p | C₈₆H₉₉N₁₀O₂₆Cl | 1722 | 1723 |
| 70 | H | OH | BnCI-p | C₈₀H₉₄N₁₀O₂₆Cl₂ | 1680 | 1681 |
| 71 | H | NH(CH₂)₄CH(C | H | C₉₀H₁₁₅N₁₂O₂₈Cl | 1846 | 1847 |
| | | ONH | | | | |
| | | (CH₂)₃NMe₂)NH | | | | |
| | | BnOBu-p | | | | |
| 72 | H | OH | Bn(PhCI-p)- | C₈₆H₉₉N₁₀O₂₆Cl₂ | 1757 | 1758 |
| | | p | | | | |
| 4 | CH₂NH(CH₂)₃N⁺ | NH(CH₂)₃NMe₂ | H | C₉₄H₁₃₆N₁₄O₂₅Cl | 1895 | 1896 |
| | Me₂C₁₀H₂₁ | | | | | |
| 5 | CH₂N[CH₂CH₂]₂ | NHMe | H | C₉₀H₁₁₆N₁₃O₂₅Cl | 1813 | 1814 |
| | NBnBu-p | | | | | |
| 73 | H | NHBnPhCI-p | H | C₈₆H₁₀₀N₁₁O₂₅Cl₂ | 1756 | 1757 |
| 74 | H | NHBnPh-p | H | C₈₆H₁₀₀N₁₁O₂₅Cl | 1721 | 1722 |
| 75 | CH₂NHBnPhCI-p | OH | H | C₈₇H₁₀₂N₁₁O₂₆Cl₂ | 1786 | 1787 |
| 76 | H | NHBnBu-p | H | C₈₄H₁₀₄N₁₁O₂₅Cl | 1701 | 1702 |
| 77 | H | NHC₇H₁₅ | H | C₈₀H₁₀₄N₁₁O₂₅Cl | 1653 | 1654 |
| 78 | CH₂N[CH₂CH₂]₂NC | OH | H | C₉₁H₁₁₅N₁₂O₂₇Cl | 1842 | 1843 |
| | OCH₂NHBnBu-p | | | | | |
| 79 | H | N[CH₂CH₂]₂NC | H | C₉₀H₁₁₃N₁₂O₂₆Cl | 1812 | 1813 |
| | | HCH₂NHBnBu-p | | | | |
| 80 | CH₂N[CH₂CH₂]₂NC | OH | H | C₈₈H₁₁₇N₁₁O₂₇Cl | 1794 | 1795 |
| | OC₉H₁₉ | | | | | |
| 81 | H | N[CH₂CH₂]₂NC | H | C₈₇H₁₁₅N₁₁O₂₆Cl | 1764 | 1765 |
| | | OC₉H₁₉ | | | | |
| 3 | CH₂NHBnBu-p | NHMe | H | C₈₆H₁₀₈N₁₂O₂₅Cl | 1727 | 1727 |
| 82 | H | NHCH((CH₂)₄N | H | C₉₀H₁₁₆N₁₃O₂₆Cl | 1829 | 1830 |
| | | H₂)CONHBnBu- | | | | |
| | | p | | | | |
| 83 | H | NHAdam-2 | H | C₈₃H₁₀₄N₁₁O₂₅Cl | 1689 | 1690 |
| 84 | CH₂NHAdam-2 | OH | H | C₈₄H₁₀₆N₁₁O₂₆Cl | 1719 | 1720 |
| 85 | CH₂NHAdam-2 | NHMe | H | C₈₅H₁₀₉N₁₂O₂₅Cl | 1732 | 1733 |
| 86 | H | COCH2NHBnO | NO | C₈₉H₁₀₃N₁₂O₂₉Cl | 1861 | 1862 |
| | | C₈H₁₇-p | | | | |
| 87 | H | CONH₂NHBnO | NO | C₉₃H₁₁₃N₁₂O₂₉Cl | 1839 | 1840 |
| | | Bn-P | | | | |

TABLE 2
Teicoplanin type glycopeptides and their derivatives.

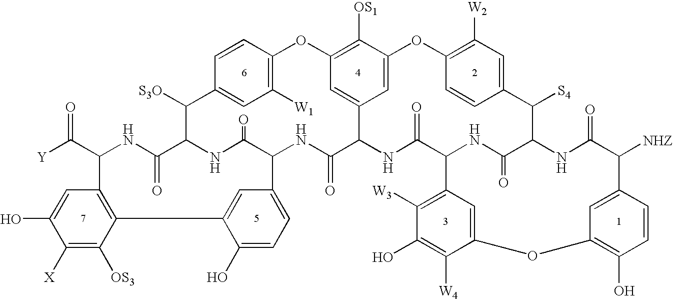

| | | | | | | |
|---|---|---|---|---|---|---|
| | | | | | | MW |
| Code | | | | | MW | [M + H} |
| no. | X | Y | Z | Brutto formula | calc | found |

Ristomycin W₁ = W₂ = W₃ = H, W₄ = Me,
S₁ = tetrasaccharide, S₂ = Ristosamine,
S₃ = S₃ = Man, S₄ = OH

| | | | |
|---|---|---|---|
| Risto | H | OMe | H |

Ristosaminylaglycon of ristomycin W₁ = W₂ = W₃ =
S₁ = S₃ = H, W₄ = Me, S₂ = Ristosamine, S₄ = OH

| | | | | | | |
|---|---|---|---|---|---|---|
| 88 | H | OMe | H | C₆₆H₆₄N₈O₂₁ | 1304 | 1305 |

Teicoplanin and its derivatives W₁ = W₂ = CI,
W₃ = S₄ = H, S₁ = GlcNAcyl, S₂ = GlcNAc,
S₃ = Man, S₄ = H

| | | | | | | |
|---|---|---|---|---|---|---|
| Teico | H | OH | H | C₈₈H₉₇N₉O₃₃Cl₂ | 2006 | 2007 |
| 89 | H | NH(CH₂)₃N⁺Me₂C₁₀H₂₁ | H | C₁₀₃H₁₃₀N₁₁O₃₂Cl₂ | 2105 | 2106 |
| 90 | H | NHMe | H | C₈₉H₁₀₀N₁₀O₃₂Cl₂ | 1893 | 1894 |

TABLE 3
N-deacyl-A40926 (DA40), demannosyl-N-deacylA40926 (DMDA40) and their derivatives.

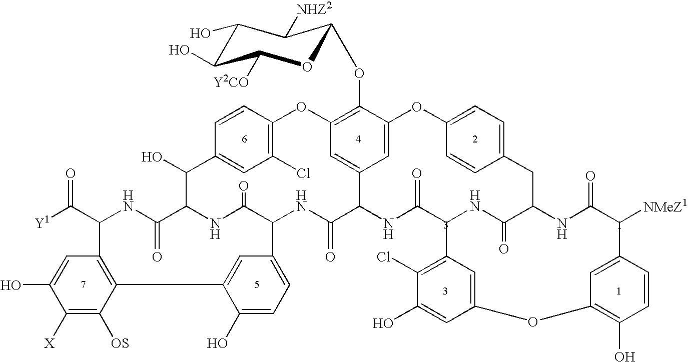

| | | | | | | | |
|---|---|---|---|---|---|---|---|
| | | | | | | | MW |
| Code | | | | | | MW | [M + 2H] |
| no. | X | Y¹ = Y² | Z¹ | Z² | Brutto formula | Calc | found |

DA4O and its derivatives S = Man

| | | | | | | | |
|---|---|---|---|---|---|---|---|
| 91 | H | OH | H | H | C₇₁H₆₆N₈O₂₈ | 1551 | 1553 |
| | | | | | Cl₂ | | |
| 10 | H | NH(CH₂)₃N⁺ | H | H | C₁₀₇H₁₁₂N₁₂O₂₆ | 2053 | 2055 |
| | | Me₂BnPh-p | | | Cl₂ | | |

DMDA4O and its derivatives S = H

| | | | | | | | |
|---|---|---|---|---|---|---|---|
| 92 | H | OH | H | H | C₆₅H₅₆N₈O₂₃Cl₂ | 1389 | 1391 |
| 11 | H | NH(CH₂)₃NMe₂ | p- | p- | C₉₇H₁₀₈N₁₂O₂₃ | 1881 | 1883 |
| | | | Bu | BuO | Cl₂ | | |
| | | | OB | Bn | | | |
| | | | n | | | | |
| 12 | H | NH(CH₂)₃NMe₂ | H | p- | C₈₆H₉₄N₁₂O₂₁ | 1703 | 1705 |
| | | | | BuB | Cl₂ | | |
| | | | | n | | | |
| 93 | CH₂N[CH₂CH₂]₂NBnPh-p | OH | H | p- | C₉₄H₉₀N₁₀O₂₃ | 1799 | 1801 |
| | | | | BuB | | Cl₂ | |
| | | | | n | | | |
| 13 | CH₂N[CH₂CH₂]₂NBnPh-p | NH(CH₂)₃NMe₂ | H | p- | C₁₀₄H₁₁₄N₁₄O₂₁ | 1967 | 1969 |
| | | | | BuB | Cl₂ | | |
| | | n | | | | | |
| 94 | CH₂N[CH₂CH₂]₂NBnBu-p | OH | H | H | C₈₁H₈₀N₁₀O₂₃ | 1633 | 1635 |
| | | | | | Cl₂ | | |
| 95 | CH₂NH(CH₂)₃N⁺C₁₀H₂₁ | OH | H | H | C₈₁H₉₁N₁₀O₂₃ | 1644 | 1646 |
| | Me2 | | | | Cl₂ | | |
| 14 | CH₂NH(CH₂)₃N⁺C₁₀H₂₁ | NH(CH₂)₃NMe₂ | H | H | C₉₁H₁₁₅N₁₄O₂₁ | 1812 | 1814 |
| | Me2 | | | | Cl₂ | | |

TABLE 4
Vancomycin type aglycons and their derivatives.

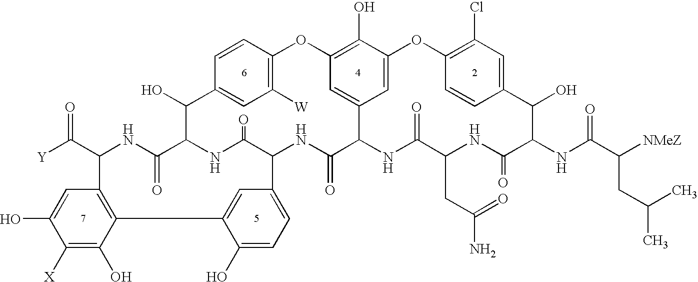

| | | | | | | |
|---|---|---|---|---|---|---|
| Code | | | | | MW | MW |
| no. | X | Y | Z | Brutto formula | Calc. | [M] found |

Vancomycin aglycon (VA) and its derivatives W = Cl

| | | | | | | |
|---|---|---|---|---|---|---|
| 96 | H | OH | H | C₅₃H₅₂N₈O₁₇Cl2 | 1143 | 1143 |

Eremomycin aglycon (EM and its derivatives W = H

| | | | | | | |
|---|---|---|---|---|---|---|
| 97 | H | OH | H | C₅₃H₅₃N₈₀O₁₇Cl | 1108 | 1108 |
| 6 | CH₂N[CH₂CH₂]₂ | OH | H | C₇₁H₇₅N₁₀O₁₇Cl | 1374 | 1374 |
| | NBnPh-p | | | | | |
| 98 | CH₂N[CH₂CH₂]₂NBn | OH | Boc | C₇₆H₈₃N₁₀O₁₉Cl | 1474 | 1474 |
| | Ph-p | | | | | |
| 7 | CH₂N[CH₂CH₂]₂NBn | NHMe | Boc | C₇₇H₈₆N₁₁O₁₈Cl | 1487 | 1487 |
| | Ph-p | | | | | |
| 8 | CH₂N[CH₂CH₂]₂NBn | NHMe | H | C₇₂H₇₈N₁₁O₁₆Cl | 1387 | 1387 |
| | Ph-p | | | | | |
| 99 | H | 1-Adam CH₂NH | H | C₆₄H₇₀N₉O₁₆Cl | 1255 | 1255 |
| 100 | H | p-FBnNH | H | C₆₀H₅₉N₉O₁₆ClF | 1215 | 1215 |
| 101 | H | (perhydroisoquinolin- | H | C₆₂H₆₈N₉O₁₆Cl | 1229 | 1229 |
| | | 1-yl)NH | | | | |
| 102 | H | 1,3- | H | C₆₆H₇₅N₁₀O₁₇Cl | 1314 | 1314 |
| | | dicyclohexylureide | | | | |
| 103 | H | 3-ethyl-1-(3- | H | C₆₁H₇₀N₁₁O₁₇Cl | 1263.5 | 1263.5 |
| | | dimethylaminopropyl | | | | |
| | | ureide + | | | | |
| | | 3-ethyl-3-(3- | | | | |
| | | dimethylaminopropyl | | | | |
| | | ureide | | | | |

Eremoniycin aglycon hexapeptide (EAH) and its derivatives W = H, first amino acid (N-Me-D-Leu) = H

| | | | | | | |
|---|---|---|---|---|---|---|
| 104 | H | OH | — | C₄₆H₄₀N₇O₁₆Cl | 981 | 981 |
| 105 | CHNHAdam-2 | OH | — | C₅₇H₅₇N₇O₁₆Cl | 1130 | 1130 |
| 9 | CH₂NHAdam-2 | NHMe | — | C₅₈H₆₀N₈O₁₅Cl | 1143 | 1143 |
| 106 | H | p-FBnNH | — | C₅₃H₄₆N₈O₁₅ClF | 1089 | 1089 |
| 107 | H | (1-Adam)CH2NH | — | C₅₇H₅₇N₈O₁₅Cl | 1129 | 1129 |
| 108 | H | (perhydroisoquinolin- | — | C₅₅H₅₅N₈O₁₅Cl | 1102 | 1102 |
| | | 1-yl)NH | | | | |
| 109 | H | OH | D-trp | C₅₇H₅₀N₉O₁₇Cl | 1167 | 1167 |

TABLE 5
Teicoplanin type aglycons and their derivatives.

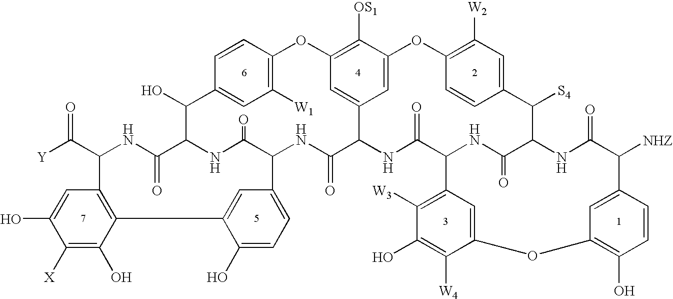

| | | | | | | | |
|---|---|---|---|---|---|---|---|
| | | | | | | | MW |
| Code | | | | | | MW | {M + H} |
| no. | X | Y | Z | S₁ | Brutto formula | calc. | found |

Ristomycin aglycon W₁ = W₂ = W₃ = H, W₄ = Me, S₄ = OH

| | | | | | | | |
|---|---|---|---|---|---|---|---|
| 110 | H | Ome | H | H | C₆₀H₅₂N₁O₁₉ | 1174 | 1175 |

Aglycon DA4O W₁ = W₃ = CI, W₄ = S₄ = H,

| | | | | | | | |
|---|---|---|---|---|---|---|---|
| 111 | H | OH | Me | H | C₅₉H₄₇N₇O₁₈Cl₂ | 1212 | 1213 |

Teicoplanin aglycon (TD) and its derivatives W₁ = W₂ = CI, W₃ =
W₄ = S₄ = H

| | | | | | | | |
|---|---|---|---|---|---|---|---|
| 112 | H | OH | H | H | C₅₈H₄₅N₇O₁₈Cl₂ | 1199 | 1200 |
| 15 | CH₂NHC₁₀H₂₁ | NH(CH₂)₃NMe₂ | H | H | C₇₄H₈₀N₁₀O₁₇Cl₂ | 1452 | 1453 |
| 16 | CH₂NH(CH₂)₄CH(NH₂₎ | NH(CH₂)₃NMe₂ | H | H | C₈₀H₉₂N₁₂O₁₈Cl₂ | 1580 | 1581 |
| | CONHC₁₀H₂₁ | | | | | | |
| 113 | H | N[CH₂CH₂]₂NN = | H | H | C₆₉H₅₇N₁₀O₁₇Cl₃ | 1404 | 1405 |
| | | CHPhCI-p | | | | | |
| 17 | CH₂N[CH₂CH₂]₂NN = | OH | H | H | C₇₀H₅₉N₁₀O₁₈Cl₃ | 1434 | 1435 |
| | CHPhCI-p | | | | | | |
| 18 | CH₂N(COLys)C₁₀H₂₁ | NH(CH₂)₃NMe₂ | H | H | C₇₂H₇₃N₉O₁₇Cl₂ | 1407 | 1408 |
| 114 | CH₂NHC₁₀H₂₁ | NH(CH₂)₃NH(CH₂)₃ | H | H | C₇₈H₉₀N₁₂O₁₇Cl₂ | 1538 | 1539 |
| | | NH(CH₂)₃NH₂ | | | | | |
| 115 | CH₂NMeBnCI-p | OH | H | H | C₆₇H₅₅N₈O₁₈Cl₃ | 1366 | 1367 |
| 116 | H | NHC₁₀H₂₁ | H | H | C₆₈H₆₆N₈O₁₇Cl₂ | 1338 | 1339 |
| 117 | CH₂NMeBnPh-p | OH | H | H | C₇₃H₆₀N₈O₁₈Cl₂ | 1408 | 1409 |
| 118 | CH₂NH(CH₂)₄CH(NH₂) | NH(CH₂)₃NMe₂ | H | H | C₇₅H₈₃N₁₃O₁₈Cl₂ | 1524 | 1525 |
| | CONH(CH₂)₃NMe₂ | | | | | | |
| 119 | CH₂N[CH₂CH₂]₂NBnCI- | OH | H | H | C₇₀H₆₀N₉O₁₈Cl₃ | 1421 | 1422 |
| | p | | | | | | |
| 20 | CH₂NH(CH₂)₃NMe₂ | NHC₁₀H₂₁ | H | H | C₇₄H₈₀N₁₀O₁₇Cl₂ | 1452 | 1453 |
| 19 | CH₂NHAdam-2 | NH(CH₂)₃NMe₂ | H | H | C₇₄H₇₄N₁₀O₁₇Cl₂ | 1446 | 1447 |
| 21 | CH₂NHC₉H₁₉ | NH(CH₂)₃NMe₂ | H | H | C₇₃H₇₈N₁₀O₁₇Cl₂ | 1436 | 1437 |
| 22 | CH₂NHC₁₀H₂₁ | NH(CH₂)₃-2-Me- | H | H | C₇₈H₈₆N₁₀O₁₇Cl₂ | 1497 | 1498 |
| | | pipecoline | | | | | |
| 120 | H | NHMe | H | H | C₅₉H₄₈N₈O₁₇Cl₂ | 1212 | 1213 |
| 23 | H | NH(CH₂)₄CH(NH₂)C | H | H | C₇₄H₇₈N₁₀O₁₈Cl₂ | 1466 | 1467 |
| | | O NHC₁₀H₂₁ | | | | | |
| 24 | CH₂NHC₁₀H₂₁ | NHMe | COLys | H | C₇₆H₈₃N₁₁O₁₈Cl₂ | 1509 | 1510 |
| 121 | CH₂NHC₁₀H₂₁ | NH(CH₂)₃NMe₂ | COLys | H | C₈₀H₉₂N₁₂O₁₈Cl₂ | 1580 | 1581 |
| 122 | H | OH | p-PhBn | H | C₇₁H₅₅N₇O₁₉Cl₂ | 1365 | 1366 |
| 123 | CH₂N[CH₂CH₂]₂NBnPh- | N[CH₂CH₂]₂NBnPh-p | p-BuBn | H | C₁₀₄H₉₇N₁₁O₁₇Cl₂ | 1843 | 1844 |
| | p | | | | | | |
| 124 | H | N[CH₂CH₂]₂NBnBu- | H | H | C₇₃H₆₇N₉O₁₇Cl₂ | 1413 | 1414 |
| | | p | | | | | |
| 125 | H | N[CH₂CH₂]₂N | H | H | C₇₃H₆₇N₉O₁₈Cl₂ | 1429 | 1430 |
| | | BnOBu-p | | | | | |
| 126 | H | N[CH₂CH₂]₂NC₁₀H₂ | H | H | C₇₂H₇₃N₉O₁₇Cl₂ | 1407 | 1408 |
| 127 | H | N[CH₂CH₂]₂N | H | H | C₇₇H₆₅N₉O₁₇Cl₂ | 1459 | 1460 |
| | | BnCHCHPh-p | | | | | |
| 25 | H | N[CH₂CH]N-2- | H | H | C₇₃H₆₁N₉O₁₇Cl₂ | 1407 | 1408 |
| | | naphtyl | | | | | |
| 26 | H | NH(CH₂)₄CH | H | H | C₈₆H₈₈N₁₁O₁₉Cl₂ | 1636 | 1637 |
| | | (NHBnOBu-p) | | | | | |
| | | CONH(CH₂)₃NMe₂ | | | | | |
| 27 | H | NH(CH₂)₃N⁺Me₂C₁₀ | H | H | C₇₃H₇₈N₉O₁₇Cl₂ | 1424 | 1425 |
| | | H₂₁ | | | | | |
| 28 | CH₂N[CH₂CH₂]₂NBnPh- | NH(CH₂)₃NMe₂ | H | H | C₈₁H₇₇N₁₁O₁₁Cl₂ | 1547 | 1548 |
| | p | | | | | | |
| 29 | | NH(CH₂)₃N⁺Me₃ | H | H | C₈₂H₈₀N₁₁O₁₇Cl₂ | 1562 | 1563 |
| 128 | H | OH | H | p-PhBn | C₇₁H₅₃N₇O₁₉Cl₂ | 1365 | 1366 |
| 129 | H | NH(CH₂)₃NMe₂ | H | C₁₁H₂₃ | C₇₄H₇₉N₉O₁₇Cl₂ | 1437 | 1438 |
| 32 | H | NH(CH₂)₃NMe₂ | C₁₁H₂₃ | H | C₇₄H₇₉N₉O₁₇Cl₂ | 1437 | 1438 |
| 30 | CH₂N[CH₂CH₂]₂NBnBu | NH(CH₂)₃NMe₂ | H | H | C₇₉H₈₁N₁₁O₁₇Cl₂ | 1527 | 1528 |
| | -p | | | | | | |
| 31 | CH₂N[CH₂CH₂]₂NBnBu | NHMe | H | H | C₇₅H₇₂N₁₀O₁₇Cl₂ | 1456 | 1457 |
| | -p | | | | | | |
| 33 | CH₂NH(CH₂)₃N⁺ | OH | H | H | C₇₄H₈₀N₉O₁₈Cl₂ | 1454 | 1455 |
| | Me2C10H21 | | | | | | |
| 34 | CH₂NH(CH₂)₃N⁺Me₂C₁₀ | NH(CH2)3NMe2 | H | H | C₇₉H₉₂N₁₁O₁₇Cl₂ | 1538 | 1539 |
| | H₂₁ | | | | | | |
| 35 | CH₂NH(CH₂)₂N⁺ | NHMe | H | H | C₇₅H₈₃N₁₀O₁₇Cl₂ | 1467 | 1468 |
| | Me₂C₁₉H₂₁ | | | | | | |
| 36 | CH₂NH(CH₂)₃N⁺ | NH(CH₂)₂OH | H | H | C₇₆H₈₅N₁₀O₁₈Cl₂ | 1497 | 1498 |
| | Me₂C₁₀H₂₁ | | | | | | |
| 37 | CH₂NH(CH₂)₃N⁺Me₂C₁₀ | NH(CH₂)₃N⁺Me₂C₁₀ | H | H | C₈₉H₁₁₃N₁₁O₁₇Cl₂ | 1679 | 1680 |
| | H₂₁ | H₂₁ | | | | | |
| 130 | H | NH(CH₂)₆NHBnBu-p | H | H | C₇₅H₇₄N₉O₁₇Cl₂ | 1442 | 1443 |
| 131 | H | OH | H | CH₂CH₂N | C₆₀H₅₀N₈O₁₈Cl₂ | 1242 | 1243 |
| | | | H₂ | | | | |
| 132 | H | OH | p-BuOBn | CH₂CH₂N | C₈₂H₇₆N₈O₂₀Cl₂ | 1564 | 1565 |
| | | | HBnOBu- | | | | |
| | | | p | | | | |
| 38 | CH₂N[CH₂CH₂]₂N⁺C₁₀ | NH(CH₂)₃NMe₂ | H | H | C₇₉H₉₀N₁₁O₁₇Cl₂ | 1536 | 1537 |
| | H₂₁ | | | | | | |
| 39 | CH₂N[CH₂CH₂]₂N⁺C₁₀ | NH(CH_{2)3N+Me2C10} | H | H | C₈₉H₁₁₁N11O₁₇Cl₂ | 1677 | 1678 |
| | H₂₁ | H₂₁ | | | | | |
| 40 | H | N[CH₂CH₂]₂NCOC₉ | H | H | C₇₂H₇₁N₉O₁₈Cl₂ | 1448 | 1449 |
| | | H₁₉ | | | | | |
| 41 | H | NH(CH₂)₆NH₂ | H | H | C₆₄H₅₉N₉O₁₇Cl₂ | 1297 | 1298 |
| 42 | CH₂NH(CH₂)₃N⁺ | NH(CH₂)₆NH₂ | H | H | C₈₀H₉₄N₁₁O₁₇Cl₂ | 1552 | 1553 |
| | Me₂C₁₀H₂₁ | | | | | | |
| 133 | H | NH(CH₂)₆NHCOEre | H | H | C₁₃₇H₁₄₉N₁₉O₄₂Cl₃ | 2838 | 2839 |
| 134 | CH₂NH(CH₂)₃N⁺ | NH(CH₂)₆NHCOEre | H | H | C₁₅₃H₁₈₃N₂₁O₄₂Cl₃ | 3092 | 3093 |
| | Me₂C₁₀H₂₁ | | | | | | |
| 135 | CH₂NH(CH₂)₃N⁺ | NH(CH₂)₆NHCOEre | H | H | C₁₆₉H₂₁₇N₂₃O₄₂Cl₃ | 3346 | 3347 |
| | Me₂C₁₀H₂₁ | CH₂NH(CH₂)₃ | | | | | |
| | | N⁺Me₂C₁₀H₂₁ | | | | | |
| 136 | CH₂NHBnBu-p | OH | H | H | C₇₀H₆₂N₈O₁₈Cl₂ | 1374 | 1375 |
| 43 | H | NH(CH₂)₁₀NH₂ | H | H | C₆₈H₆₇N₉O₁₇Cl₂ | 1353 | 1354 |
| 137 | H | NHBnNBu₂-p | H | H | C₇₃H₇₀N₉O₁₇Cl₂ | 1416 | 1417 |
| 138 | H | NH(CH₂)₅CO-D-Ala- | H | H | C₇₁H₆₅N₁₀O₂₁Cl₂ | 1454 | 1455 |
| | | D-Ala | | | | | |
| 44 | H | NH(CH₂)₅CO-D-Ala- | Boc | H | C₇₅H₇₄N₁₀O₂₃Cl₂ | 1554 | 1555 |
| | | D-Ala | | | | | |
| 139 | CH₂NHMe | OH | H | H | C₆₀H₅₀N₈O₁₈Cl₂ | 1242 | 1243 |
| 140 | CH₂NHMe | OH | Boc | H | C₆₄H₅₈N₈O₂₀Cl₂ | 1342 | 1343 |
| 45 | CH₂NHMe | NHMo | H | H | C₆₁H₅₃N₉O₁₇Cl₂ | 1255 | 1256 |
| 141 | CH₂N[CH₂CH₂]₂NCOC₉ | OH | H | H | C₇₃H₇₃N₉O₁₉Cl₂ | 1492 | 1493 |
| | H₁₉ | | | | | | |
| 142 | CH₂N[CH₂CH₂]₂NCO | OH | H | H | C₇₆H₇₂N₁₀O₁₉Cl₂ | 1500 | 1501 |
| | CH₂NHBnBu-p | | | | | | |
| 46 | H | N[CH₂CH₂]₂N | H | H | C₇₅H₇₀N₁₀O₁₈Cl₂ | 1470 | 1471 |
| | | COCH₂NHBnBu- | | | | | |
| 143 | CH₂N[CH₂CH₂]₂NCOC | NHMe | H | H | C₇₇H₇₅N₁₁O₁₈Cl₂ | 1513 | 1514 |
| | H₂NHBnBu-p | | | | | | |
| 144 | CH₂N[CH₂CH₂]₂N | NHMe | H | H | C₇₄H₇₆N₁₀O₁₈Cl₂ | 1505 | 1506 |
| | COC₉H₁₉ | | | | | | |
| 145 | H | 6-APA | H | H | C₆₆H₅₅N₁₀O₂₂Cl₂S | 1397 | 1398 |
| 146 | CH₂NHBnBu-p | OH | Boc | H | C₇₅H₇₀N₈O₂₀Cl₂ | 1474 | 1475 |
| 47 | CH₂NHBnBu-p | NHMe | Boc | H | C₇₆H₇₃N₉O₁₉Cl₂ | 1487 | 1488 |
| 48 | CH₂NHBnBu-p | NHMe | H | H | C₇₁H₆₅N₉O₁₇Cl₂ | 1387 | 1388 |
| 147 | H | OH | Boc | H | C₆₃H₅₃N₇O₂₀Cl₂ | 1299 | 1300 |
| 148 | H | OH | Fmoc | H | C₇₃H₅₅N₇O₂₀Cl₂ | 1421 | 1422 |
| 49 | H | OH | Adoc | H | C₆₉H₅₉N₇O₂₀Cl₂ | 1377 | 1378 |
| 149 | H | OH | Cbz | H | C₆₆H₅₁N₇O₂₀Cl₂ | 1333 | 1334 |
| 150 | H | NHAdam-2 | Boc | H | C₇₃H₆₈N₈O₁₉Cl₂ | 1432 | 1433 |
| 151 | H | NHMe | Boc | H | C₆₄H₅₆N₈O₁₉Cl₂ | 1312 | 1313 |
| 152 | H | NHMe | Adoc | H | C₇₀H₆₂N₈O₁₉Cl₂ | 1390 | 1391 |
| 153 | H | OH | C(S)NHPh | H | C₆₅H₅₀N₈O₁₈Cl₂S | 1334 | 1335 |
| 50 | H | NHAdam | H | H | C₆₈H₆₀N₈O₁₇Cl₂ | 1332 | 1333 |
| 154 | CH₂NHAdam-2 | OH | H | H | C₆₉H₆₂N₈O₁₈Cl₂ | 1362 | 1363 |
| 51 | CH₂NHAdam-2 | NHMe | H | H | C₇₀H₆₅N₉O₁₇Cl₂ | 1375 | 1376 |
| 155 | CH₂NHC₁₂H₂₅ | OH | H | H | C₇₁H₇₂N₈O₁₈Cl₂ | 1396 | 1397 |
| 156 | CH₂NHC₁₂H₂₅ | NHMe | H | H | C₇₂H₇₅N₉O₁₇Cl₂ | 1409 | 1410 |
| 157 | CH₂NHC₁₈H₃₇ | OH | H | H | C₇₇H₈₄N₈O₁₈Cl₂ | 1480 | 1481 |
| 158 | CH₂NHC₁₈H₃₇ | NHMe | H | H | C₇₈H₈₇N₉O₁₇Cl₂ | 1493 | 1494 |
| 52 | CH₂NHAdam-2 | NHAdam-2 | H | H | C₇₉H₇₇N₉O₁₇Cl₂ | 1495 | 1496 |
| 159 | H | OH | H | H | C₅₈H₄₅N₇O₁₈Cl₂ | 1199 | 1200 |
| 160 | H | (1- | H | H | C₇₀H₆₄N₈O₁₇Cl₂ | 1359 | 1360 |
| | H | Adam)CH(CH₃)NH | | | | | |
| 161 | H | (perhydroisoquinolin- | H | H | C₆₇H₆₀N₈O₁₇Cl₂ | 1318 | 1319 |
| | | 1-yl)NH | | | | | |
| 162 | H | (2-exo-norbornyl)NH | H | H | C₆₅H₅₆N₈O₁₇Cl₂ | 1290 | 1291 |
| 163 | H | OH | (glyoxalyl- | H | C₆₈H₅₀N₈O₂₀Cl₂ | 1368 | 1369 |
| | | | indol-3-yl)- | | | | |
| 164 | H | OH | 1- | H | C₆₉H₆₆N₇O₁₉Cl₂ | 1360 | 1361 |
| | | | adamantoy | | | | |
| | | | 1- | | | | |
| 165 | H | p-FBnNH | H | H | C₆₅H₅₁N₈O₁₇Cl₂ | 1304 | 1305 |
| 166 | H | (1-Adam)CH₂NH | H | H | C₆₉H₆₂N₈O₁₇Cl₂ | 1344 | 1345 |
| 167 | H | 1,3- | H | H | C₇₁H₆₇N₉O₁₈Cl₂ | 1403 | 1404 |
| | | dicyclobexylureide | | | | | |
| 168 | H | 3-ethyl-1-(3- | H | H | C₆₅H₆₂N₁₀O₁₈Cl₂ | 1340 | 1341 |
| | | dimethylaminopropy | | | | | |
| | | 1-ureide + | | | | | |
| | | 3-ethyl-3-(3- | | | | | |
| | | dimethylaminopropy | | | | | |
| | | 1-ureide | | | | | |

TABLE 6
Teicoplanin aglycon derivatives with eliminated amino acids 1 and 3

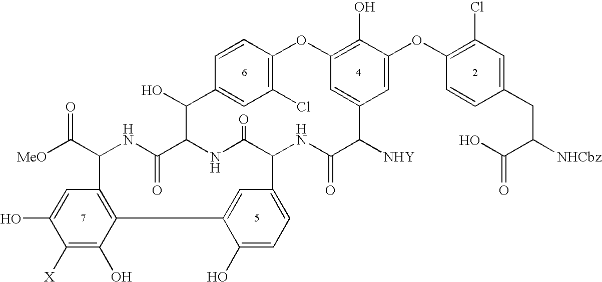

| | | | | | MW found |
|---|---|---|---|---|---|
| Code no. | X | Y | Brutto formula | MW calc | [M] |
| 169 | H | H | C₅₁H₄₃N₅O₁₆Cl₂ | 1053 | 1053 |
| 170 | H | Boc | C₅₆H₅₁N₅O₁₈Cl₂ | 1152 | 1152 |
| 53 | CH₂NHAdam-2 | Boc | C₆₇H₆₈N₆O₁₈Cl₂ | 1315 | 1315 |
| 54 | CH₂NHAdam-2 | H | C₆₂H₆₀N₆O₁₆C1₂ | 1215 | 1215 |

TABLE 7
Teicoplanin aglycon derivatives with the disrupted bond betweem amino acids 1 and 2

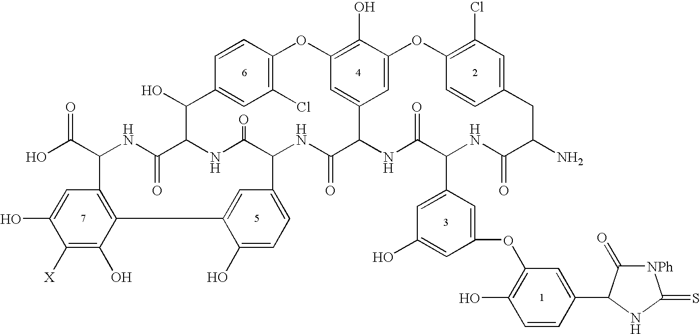

| Code no. | X | Brutto formula | MW calc | MW [M] found |
|---|---|---|---|---|
| 171 | H | C₆₅H₅₁N₈O₁₈Cl₂S | 1335 | 1335 |
| 55 | CH₂NHAdam-2 | C₇₆H₆₈N₉O₁₈Cl₂S | 1498 | 1498 |

TABLE 8
Teicoplanin aglycon with the disrupted bond between amino acids 6 and 7

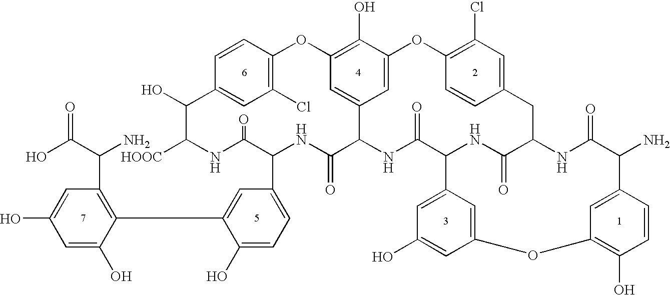

| Code no. | Brutto formula | MW calc | MW found [M + H] |
|---|---|---|---|
| 172 | C₅₈H₄₇N₇O₁₉Cl₂ | 1217 | 1218 |

Footnote: Adam-1 = adamant-1-yl, adam-2 = adamant-2-yl

### Example 2

### General Methods and Materials for the Preparation of the Compounds

The glycopeptide antibiotics and their derivatives and more particularly the compounds of formula Z or I, II and III of this invention can be prepared while using a series of chemical reactions well known to those skilled in the art, altogether making up the process for preparing said compounds and exemplified further. The processes described further are only meant as examples and by no means are meant to limit the scope of the present invention.

The compounds of the invention can conveniently be prepared by following (one of) the methods described below. All the compounds shown in tables 1 to 8 were prepared by following these methods of preparation.

All reagents and solvents can be purchased from Aldrich (Milwaukee), Fluka (Deisenhofen, Germany), Sigma Corporation (St. Louis, Mo.) and Merck (Darmstadt, Germany). The novel compounds were obtained by applying methods (e.g. amidation, Mannich reaction, N-acylation) previously described for the synthesis of other glycopeptide derivatives.

Method A. Aminomethylated derivatives (i.e. 1, 6, 24, 51, 52, 53, 54, 55, 61-64, 67, 75, 78, 80, 84, 85, 93, 94, 95, 98, 105, 115, 117, 119, 121, 122, 154, 155, 156, 157, 158)

To a stirred solution of 0.5 mmol of antibiotic or its degradation product and 4 mmol of an appropriate amine in 10 ml of an acetonitrile-water 1:1 mixture was added 3 mmol of 37% aqueous formaldehyde. If a salt of amine was used in NaOH was added to pH 10. The reaction mixture was stirred at room temperature for 18 h and then 100 ml of water was added. After adjusting the reaction mixture at pH 3 with In HCl, the resulting solution (or suspension) was extracted with n-BuOH (˜25 ml×2); the organic layer was washed with water (˜15 ml×2) and then concentrated at 45° C. in a vacuum to a small volume (˜3 ml). On adding ether (˜100 ml), the precipitated solid was collected and dried in vacuum at room temperature for 4 h. Then it was dissolved in a minimal amount of MeOH and applied to a chromatographic column with Sephadex LH-20 (2×100 cm) preequilibrated with MeOH. The column was developed with MeOH at a rate of 10 ml/h, while collecting 5 ml fractions. The suitable fractions were combined and concentrated to a small volume (˜3 ml). After adding ether (˜100 ml) the precipitate formed was collected, rinsed with ether and dried in vacuum at room temperature. The starting compound for 53—N²-Cbz-N⁴-Boc-TDTP-Me—was obtained as previously described. Compound 54 was obtained from 53 by the removal of Boc-group in TFA as previously described for N²-Cbz-N⁴-Boc-TDTP-Me (Malabarba, A.; Ciabatti, R.; Maggini, M.; Ferrari, P.; Vekey, K.; Colombo, L.; Denaro, M. Structural modifications of the active site in teicoplanin and related glycopeptides.2. Deglucoteicoplanin-derived tetrapeptide. J. Org. Chem. 1996, 61, 2151-2157).

The starting compound for 55—N-terminal phenylthiohydantoin-derivative of teicoplanin aglycon—was obtained by Edman degradation of teicoplanin aglycon.

Method B. Carboxamides (i.e 2, 10, 11, 12, 23, 25, 26, 27, 29, 40, 41, 43, 46, 50, 56, 57, 60, 65, 66, 71, 73, 74, 76, 77, 81, 82, 83, 89, 90, 99, 100, 101, 102, 103, 106-108, 113, 116, 120, 124-127, 137-138, 145, 150, 160, 161, 162, 165, 166, 167)

To a mixture of an antibiotic or its degradation product (0.5 mmol) and 5 mmol of an amine hydrochloride dissolved in 5 ml of DMSO were added portion-wise Et₃N to adjust pH 8.5-9 and afterwards during 1 hour 1 mmol of PyBOP-reagent (benzotriazol-1-yloxy)-tris-(pyrrolidino)phosphonium-hexafluorophosphate) or HBPyU-reagent (O-(benzotriazol-1-yloxy)-N,N,N′,N′-bis(tetramethylene)uronium hexafluorophosphate). The reaction mixture was stirred at room temperature for 3 hours.

Addition of ether (˜100 ml) to the reaction mixture led to an oily residue, which was shaken successively with ether (15 ml×2) and acetone (˜15 ml). After addition of 100 ml of acetone a precipitate of crude amide was collected, dissolved in 50 ml of water and 1 n NaOH was added to pH 9. The resulting solution (or suspension) was extracted with n-BuOH (˜25 ml×3); the organic layer was washed with water (˜15 ml×3) and then concentrated at 45° C. in vacuum to a small volume (˜3 ml). On adding ether (˜100 ml), the precipitated solid was collected and dried in a vacuum at room for 4 h. and 100 ml of acetone was added to form the precipitate, which was collected to give a pure carboxamide.

Method C. Carboxamides of Aminomethylated Derivatives (i.e. 3, 4, 5, 8, 9, 14, 15, 16, 17, 18, 19, 20, 21, 22, 28, 30, 31, 34, 35, 36, 37, 38, 39, 42, 45, 48, 51, 52)

These compounds were obtained by the method B starting from the aminometylated derivatives obtained by the method A.

Method D. N-carbamoylated Derivative. (i.e. 49, 98, 7, 147, 149, 149, 170)

To a stirred solution of 0.5 mmol of antibiotic or its degradation product in 15 ml THF-water 1:1 mixture adjusted to pH 10 with 1 n NaOH 0.55 mmol of adamantyloxycarbonyl chloride was added. The reaction mixture was stirred at room temperature for 4 h, then it was diluted with 100 ml of water. After adjusting the reaction mixture at pH 3 with In HCl, the resulting solution (or suspension) was extracted with n-BuOH (˜25 ml×2); the organic layer was washed with water (˜15 ml×2) and then concentrated at 45° C. in vacuum to a small volume (˜3 ml). On adding ether (˜100 ml), the precipitated solid was collected and dried in vacuum at room temperature for 4 h.

Method E. N-(D-Trp)-(de-N-Me-D-Leu)eremomycin aglycon (i.e. 109)

Compound 109 was obtained as previously described (Miroshnikova, O. V.; Berdnikova, T. F.; Olsufyeva, E. N.; Pavlov, A. Y.; Reznikova, M. I.; Preobrazhenskaya, M. N.; Ciabatti, R.; Malabarba, A.; Colombo, L. A Modification of the N-Terminal Amino Acid in the Eremomycin Aglycone. J. Antibiot. 1996, 49, 1157-1161).

Method F. N-carbamoylated Derivative of Carboxamide (i.e. 44)

This compound was obtained by the method D using Boc₂O reagent starting from carboxamide obtained by the method B.

Method G. N-carbamoylated Derivative of Carboxamides of Aminomethylated Derivatives (i.e. 7, 24, 47)

These compounds were obtained by the method D using Boc₂O reagent starting from carboxamides of aminomethylated derivatives obtained by the method C.

Method H. N- or N,N′-alkylated Derivatives (i.e. 11, 12, 13, 32)

To a stirred solution of 0.5 mmol of the starting compound [ethylaminopiperazinamide of DMDA 40926, obtained by the method B for compound 12; 7d-methyl-N(p-phenylbenzyl)piperazine of di-ethylaminopropylamide of DMDA40 for compound 13; 7d-methylaminobuthyl-N(nonyldimethyl)-amine of di-dimethylaminopropylamide of teicoplanin aglycone obtained by the method C for compound 32], 1.5 mmol of the corresponding aldehyde was added and the reaction mixture was stirred at 40° C. for 3 h. Then the reaction mixture was cooled to 20° C. and 1 mmol of NaCNBH₃ was added. After stirring at 20° C. for 1 h 150 ml of ether was added to the reaction mixture to give an oily residue, which was shaken successively with ether (15 ml×2) and acetone (˜15 ml). After addition of 100 ml of acetone, a precipitate of crude amide was collected, dissolved in 50 ml of water and 1 n NaOH was added to pH 9. The resulting solution (or suspension) was extracted with n-BuOH (˜25 ml×3); the organic layer was washed with water (˜15 ml×3) and then concentrated at 45° C. in vacuum to a small volume (˜3 ml). On adding ether (˜100 ml), the precipitated solid was collected and dried in vacuum at room for 4 h. and 100 ml of acetone was added to form the precipitate, which was collected to give a pure product.

The methods for introducing chemical modifications in the sugar moieties of the glycopeptide antibiotic derivatives, at the amide part, at the resorcinol fragment and at the N-end of the antibacterial glycopeptide antibiotics were elaborated earlier, and used for the preparation of a variety of semisynthetic glycopeptides (Malabarba, A.; Nicas, T. I. and Thompson, R. S. Structural Modifications of Glycopeptide Antibiotics. Med. Res. Rev. 1997, 17, 69-137; Pavlov, A. Y.; Preobrazhenskaya, M. N. Chemical Modification of Glycopeptide Antibiotics. Russian Journal of Bioorganic Chemistry 1998, 24, 570-587). Changing the nature of the sugar residues of the glycopeptide antibiotics such has vancomycin can be performed as described in Nicas, T. I. et al. (Antimicrobial agents and Chemotherapy, 1996, 40, 2194-2199.)

Degradation products, the aglycon antibiotics can be obtained through chemical degradation as described as examples hereunder.

Eremomycin aglycon was obtained as described in Berdnikova, T. F. et al (Berdnikova, T. F.; Lomakina, N. N.; Olsufyeva, E. N.; Alexandrova, L. G.; Potapova, N. P.; Rozinov, B. V.; Malkova, I.V.; Orlova, G. I. Structure and Antimicrobial Activity of Products of Partial Degradation of Antibiotic Eremomycin. Antibiotics and Chemotherapy (Rus) 1991, 36, 28-31). 1000 mg (0.6 mmol) of eremomycin sulfate were dissolved in 20 ml of HCl (concentrated) and were kept at a room temperature for 5 h. Then 60 ml of water were added to precipitate eremomycin aglycon. The mixture was cooled to 5° C. and kept in refrigerator for 3 h. The solid was filtered off, washed with 10 ml of cool water, then with aceton and dried in vacuum. The solid was dissolved in 6 ml of DMSO and was added to 60 ml of acetone. The precipitate was filterred off, washed with aceton and dried to yield 530 mg of a crude eremomycin aglycon. The water filtrate was passed through column (2×10 cm) of Dowex 50×2 resin (H⁺-form), which was washed with water and eluted with 50 ml of 0.25 N NH₄OH. The eluates were concentrated in vacuum with n-BuOH to minimal volume and precipitated with 50 ml acetone. The precipitate was collected, washed with acetone and dried in vacuum to give a crude eremomycin aglycon. The samples were analyzed by TLC on the Merck Silica Gel 60F₂₅₄ plates in systems EtOAc-PrOH-25% NH₄OH 2:2:3 with UV control.

The solids were combined and dissolved in 10 ml of 0.05 M AcONH₄-EtOH 9:1 mixture while acidified with 2 N HCl to pH 3 and applied to a chromatographic column with CM 32 carboxymethyl cellulose (Whatman, Greate Britane) (45 cm×2 cm) preequilibrated with 0.05 M AcONH₄-EtOH 9:1 mixture (pH 6.7). The column chromatography was carried out with 0.05 M AcONH₄-EtOH 9:1 mixture (pH 6.7) (300 ml), 0.1 M AcONH₄-EtOH 9:1 mixture (pH 6.7) (700 ml), then 0.15 M AcONH₄-EtOH 9:1 mixture (pH 6.7) (700 ml) at a flow rate 30 ml/h. The fractions containing eremomycin aglycon were combined, acidified with 6 N HCl to pH 3 and passed through column (2×10 cm) of Dowex 50×2 resin (H⁺-form), which was washed with water and eluted with 50 ml of 0.25 N NH₄OH. The eluates were concentrated in vacuum with n-BuOH to minimal volume, acidified with 0.05 N HCl to pH 5 and precipitated with 50 ml acetone. The precipitate was collected, washed with acetone and dried in vacuum to give 310 mg (0.28 mmol) oferemomycin aglycon (46.7%).

Des-(N-methyl-D-leucyl) eremomycin aglycon was obtained from eremomycin aglycon as described in Miroshnikova, O. V. et al. (Miroshnikova, O. V.; Berdnikova, T. F.; Olsufyeva, E. N.; Pavlov, A. Y.; Reznikova, M. I.; Preobrazhenskaya, M. N.; Ciabatti, R.; Malabarba, A.; Colombo, L. A Modification of the N-Terminal Amino Acid in the Eremomycin Aglycone. J. Antibiot. 1996, 49, 1157-1161.

Teicoplanin aglycon was obtained as described in Malabarba, A. et al. (Malabarba, A.; Ferrari, P.; Gallo, G. G.; Kettenring, J.; Cavalleri, B. Teicoplanin, Antibiotics from Actinoplanes teichomyceticus nov. sp. VII. Preparation and NMR Characteristics of the Aglycone of Teicoplanin. J. Antibiotics 1986, 39, 1430-1442). The starting compound N-terminal phenylthiohydantoin-derivative of teicoplanin aglycon, was obtained by Edman degradation of teicoplanin aglycon. To a solution of teicoplanin aglycon (100 mg, ˜0.08 mmol) in a mixture of Py/H₂O (6:1, 4 mL), triethyl amine (0.26 mL, 2 mmol) and PhNCS (0.02 mL, ˜0.16 mmol) were added at room temperature under argon. The reaction mixture was stirred for 16 h, then 8 mL of H₂O were added and the reaction mixture was evaporated with n-BuOH to dryness. The precipitate was dissolved in the mixture of TFA-CH₂Cl₂, 1:1 (3 mL) at 0-5° C. and then was stirred at this temperature for 1 h. Water (3 mL) was then added and the mixture was neutralized with 25% NH₄OH, washed with EtOAc (3 mL×3), and the aqueous fraction was concentrated in vacuum with the addition of n-BuOH and applied to a column of silanized silica gel (2×100 cm), previously equilibrated with 0.01M acetic acid. The column was eluted with acetic acid (0.01M) at a flow rate of 30 mL/h for elution of compound N-terminal phenylthiohydantoin-derivative of teicoplanin aglycon. Fractions were pooled, concentrated with the addition of n-BuOH in vacuum, and acetone (50 mL) was added to yield the precipitate, which was filtered off, washed with acetone and dried to yield 68 mg (54%).

The homogeneity, purity and identity of the compounds obtained was assessed by HPLC and ESI mass-spectrometry. Analytical reverse phase HPLC was carried out on a Shimadzu HPLC instrument of the LC 10 series on a Diasorb C16 column (particle size 7 μm) at an injection volume of 10 μL and a wavelength 280 nm. The sample concentration was 0.05-0.2 mg/mL. Two systems were used to control the final compounds: System A comprised of 0.1 M NH₄H₂PO₄ at pH 3.75 and acetonitrile, the proportion of acetonitrile increased linearly from 15 to 40% within 15 min and then the ratio of acetonitrile was kept constant during 25 min with a flow rate of 1.0 mL/min. System B comprised of 0.2% HCOONH₄ and 45% acetonitrile, with a flow rate of 0.07 mL/min.Mass spectra were determined by Electrospray Ionisation (ESI) on a Finnigan SSQ7000 single quadrupole mass spectrometer. For all the compounds presented ESI-mass spectral data correspond to the calculated values.

Analogous compounds are synthesized in the same fashion as exempligied in the foregoing methods by varying the starting material, intermediates, solvents and conditions as will be known by those skilled in the art.

### Example 3

### Methodology for Determination of Antiviral (HIV, BVDV, HCV, HSV, VZV, CMV, FCV, SARS) and Cytostatic Activity

Anti-HIV Activity Assays

Inhibition of HIV-1 (III_{B}, HE, HN) and HIV-2(ROD, EHO, RF)-induced cytopathicity in CEM or C8166 or Molt4/C8 cells was measured in microtiter 96-well plates containing ˜3×10⁵ CEM cells/ml, infected with 100 CCID₅₀ of HIV per ml and containing appropriate dilutions of the test compounds. After 4 to 5 days of incubation at 37° C. in a CO₂-controlled humidified atmosphere, CEM, C8166 or Molt4/C8 giant (syncytium) cell formation was examined microscopically. The EC₅₀ (50% effective concentration) was defined as the concentration of compound required to inhibit HIV-induced giant cell formation by 50%.

Cytostatic Activity Assays

All assays were performed in 96-well microtiter plates. To each well were added 5-7.5×10⁴ cells and a given amount of the test compound. The cells were allowed to proliferate for 48 h (murine leukemia L1210) or 72 h (human lymphocyte CEM and Molt4/clone 8) at 37° C. in a humidified CO₂-controlled atmosphere. At the end of the incubation period, the cells were counted in a Coulter counter. The IC₅₀ (50% inhibitory concentration) was defined as the concentration of the compound that reduced the number of cells by 50%.

Anti-BVDV Assay

Cells and viruses: Madin-Darby Bovine Kidney (MDBK) cells were maintained in Dulbecco's modified Eagle medium (DMEM) supplemented with BVDV-free 5% fetal calf serum (DMBM-FCS) at 37° C. in a humidified, 5% CO₂ atmosphere. BVDV-1 (strain PE515) was used to assess the antiviral activity in MDBK cells. Vero cells (ATCC CCL81) were maintained in MEM medium supplemented with 10% inactivated calf serum, 1% L-glutamine and 0.3% bicarbonate.

Anti-BVDV assay: Ninety-six-well cell culture plates were seeded with MDBK cells in DMEM-FCS so that cells reached 24 hr later confluency. Then medium was removed and serial 5-fold dilutions of the test compounds were added in a total volume of 100 ul, after which the virus inoculum (100 ul) was added to each well. The virus inoculum used resulted in a greater than 90% destruction of the cell monolayer after 5 days incubation at 37° C. Uninfected cells and cells receiving virus without compound were included in each assay plate. After 5 days, medium was removed and 90 μt of DMEM-FCS and 10 ul of MTS/PMS solution (Promega) was added to each well. Following a 2 hr incubation period at 37° C. the optical density of the wells was read at 498 nm in a microplate reader. The 50% effective concentration (EC₅₀) value was defined as the concentration of compound that protects 50% of the cell monolayer from virus-induced cytopathic effect.

Anti-HCV Assay/Replicon Assay

Huh-5-2 cells [a cell line with a persistent HCV replicon 13891uc-ubi-neo/NS3-3′/5.1; replicon with firefly luciferase-ubiquitin-neomycin phosphotransferase fusion protein and EMCV-IRES driven NS3-5B HCV polyprotein] van be cultured in RPMI medium (Gibco) supplemented with 10% fetal calf serum, 2 mM L-glutamine (Life Technologies), 1× non-essential amino acids (Life Technologies); 100 IU/ml penicillin and 100 ug/ml streptomycin and 250 ug/ml G418 (Geneticin, Life Technologies). Cells can be seeded at a different densities, particularly in a density of 7000 cells per well in 96 well View Plate™ (Packard) in medium containing the same components as described above, except for G418. Cells than can be allowed to adhere and proliferate for 24 hr. At that time, culture medium can be removed and serial dilutions of the test compounds can be added in culture medium lacking G418. Interferon alfa 2a (500 IU) can be included as a positive control. Plates can further be incubated at 37° C. and 5% CO₂ for 72 hours. Replication of the HCV replicon in Huh-5 cells results in luciferase activity in the cells. Luciferase activity is measured by adding 50 μl of 1×Glo-lysis buffer (Promega) for 15 minutes followed by 50 μl of the Steady-Glo Luciferase assay reagent (Promega). Luciferase activity can be measured with a luminometer and the signal in each individual well is expressed as a percentage of the untreated cultures. Parallel cultures of Huh-5-2 cells, seeded at a density of 7000 cells/well of classical 96-well cel culture plates (Becton-Dickinson) can be treated in a similar fashion except that no Glo-lysis buffer or Steady-Glo Luciferase reagent is added. Instead the density of the culture can be measured by means of the MTS method (Promega).

Anti-Coxsackie Virus Assay

Ninety-six-well cell culture plates can be seeded with Vero cells in DMEM medium containing 10 fetal calf serum (FCS) so that cells reache confluency 24-48 hr later. Medium can then be removed and serial 5-fold dilutions of the test compounds can be added in a total volume of 100 ul, after which the virus inoculum (100 μl) can be added to each well. The virus inoculum used results normally in a 90-100% destruction of the cell monolayer after 5 days incubation at 37° C. Uninfected cells and cells receiving virus without compound can be included in each asay plate. After 5 days, the medium can be removed and 90 μl of DMEM-FCS and 10 μl of MTS/PMS solution (Promega) was added to each well. Following a 2 h incubation period at 37° C., the optical density of the wells can be read at 498 nm in a microplate reader. The 50% effective concentration (EC50) value can than be defined as the concentration of compound that protects 50% of the cell monolayer from virus-induced cytopathic effect.

Anti-Herpes Simplex Virus, Varicella-Zoster Virus and Cytomegalovirus Assays

The antiviral assays HSV-1, HSV-2, VZV, CMV were based on inhibition of virus-induced cytopathicity in HEL cell cultures. Confluent cell cultures in microtiter 96-well plates were inoculated with 100 CCID₅₀ of virus, 1 CCID₅₀ being the virus dose required to infect 50% of the cell cultures. After a 1- to 2-h virus adsorption period, residual virus was removed, and the cell cultures were incubated in the presence of varying compound concentrations of the test compounds. Viral cytopathicity was recorded as soon as it reached completion in the control virus-infected cell cultures that were not treated with the test compounds.

Feline Corona Virus Assay

Feline Crandel kidney cells were seeded in 96-well microtiter plates at 24,000 cells/well. Then, 24 hrs later, an appropriate inoculum of FCV is added together with 5-fold dilutions of the test compounds. After 4 days, a MTS/PMS solution was added to each well. Following a 90 min incubation period at 37° C., the optical density of the wells was read at 498 nm in a microplate reader.

SARS Virus Assay

Vero cells were seeded in 96-well microtiter plates and grown till confluency. Then, an appropriate inoculum of SARS virus able to kill the cell culture (cytopathicity) within 72 hrs is added together with 5-fold dilutions of the test compounds. After 3 days, a MTS/PMS solution was added to each well. Following a 3 hr incubation period at 37° C. the optical density of the wells was read at 498 nm in a microplate reader.

### Example 4

### Evaluation of the Anti-HIV Activity of the Compounds of the Invention

A variety of glycopeptide antibiotic derivatives of vancomycin, eremomycin and teicoplanin including their aglycon derivatives were evaluated for their inhibitory activity against HIV-1 (III_{B}) and HIV-2(ROD) in CEM cell cultures.

The vancomycin derivatives 1 and 2 for example were inhibitory to HIV-1 at an EC₅₀ of 5.5 and 12 μM, respectively. The eremomycin derivative 5 proved very inhibitory to HIV-1 replication (EC₅₀: 0.43 μM) being cytotoxic against the CEM cells at a 100-fold higher concentration (IC₅₀: 40 M).

As another example, the eremomycin aglycon derivatives 6 to 8 all invariably inhibited both HIV-1 and HIV-2 at EC₅₀ values ranging between 3.5 and 12 μM. This is at compound concentrations that were at least 15- to 20-fold lower than required for the eremomycin aglycon. They were non-toxic (IC₅₀>100 μM for CEM cells). The Des-(N-methyl-D-leucyl)-eremomycin aglycon 9 was also active against IV (13-20 μM) and not toxic at 250 μM (Scheme 1, Table 1).

Further examples are antibiotic A40 926 derivatives 10 to 14 containing no N′-acyl substituent and mannose moiety at ring 6 which also displayed anti-HIV-1 activity between 3.5 and 12 μM. Other examples are the teicoplanin aglycon derivatives which showed pronounced anti-HIV-1 and anti-HIV-2 activity, often with a trend of being slightly more active against HIV-1 than HIV-2. The most active congeners were inhibitory against HIV-1 in the range of 1.3 to 4.5 μM (compounds 15, 19, 21, 22, 25, 27, 31, 23, 35-40, 42 and 52). A number of them, i.e. 52, 31, 19, 15 were not cytotoxic at 100-500 μM. This means that the most selective compounds 19 and 31 had selectivity indices (ratio IC₅₀/EC₅₀) that were ≧200. The teicoplanin aglycon showed also anti-HIV activity, but the derivatives showed an improved activity over the unsubstituted teicoplanin aglycon (EC₅₀: 17-20 μM; IC₅₀: >500 μM).

Further examples comprise compounds 53 and 54 that lack the ring systems 1 and 3 and have only two macroring structures showed activity against HIV-1 and HIV-2 at an EC₅₀ between 17 and 37 μM. Also, compound 55 showed an antiviral activity of 13 and 17 μM against HIV-1 and HIV-2, respectively

It is clear that in general, the aglycon derivatives of vancomycin, eremomycin and teicoplanin gain anti-HIV activity compared to their glycosylated parent compounds. Also, substituents on the aglycons of vancomycin, eremomycin and teicoplanin that increase the lipophilicity of the aglycon derivatives, also markedly increase the anti-HIV activity of the compounds. In some cases, just the simple aglycon showed already measurable anti-HIV activity, but hydrophobic derivatives were, as a rule, markedly more (10- to 100-fold) inhibitory to HIV. Among the teicoplanin derivatives, both low hydrophobic and highly hydrophobic compounds showed prominent anti-HIV activity.

Seven compounds (6, 19, 30, 31, 35, 46 and 51) were evaluated against a variety of HIV strains in different cell lines, and it was found that they all maintained a similar antiviral potency regardless of the nature of the cell line or virus strain.

A time of addition experiment was performed for the highly selective compound 30. Compound 30, like the virus adsorption inhibitor dextran sulfate, cannot be added later than 1 h post infection without significant loss of antiviral activity. In contrast, administration of a reverse transcriptase inhibitor (AZT, zidovudine) could be delayed for at least 3 hours without loosing its antiviral activity. A very early event in the replication (infection) cycle of HIV is the antiviral target for these glycopeptide antibiotics and novel antibiotic derivatives. In agreement with these observations, it is an important noting that the compounds kept their antiviral efficacy against HIV-1 strains that contain mutations in the reverse transcriptase that result in resistance to non-nucleoside reverse transcriptase inhibitors (NNRTIs).

Extensive attempts (≧9 weeks) to select resistant virus strains against 15, 19 and 35 failed under experimental conditions that easily resulted in the emergence of nucleoside RT inhibitors (NRTI)- (i.e. lamivudine) or NNRTI- (i.e. nevirapine) resistant virus strains.

In conclusion, novel classes of modified antibiotics have been discovered that were active and selective against mV in cell culture. The most active members of these antibiotic derivatives had an EC₅₀ of 1-3 μM and were non-toxic in cell culture (IC₅₀≧200-500 μM). Their antiviral mechanism of action is located at an early event in the infection cycle of HIV (most likely adsorption and/or fusion), and is clearly different from the molecular mechanism of anti-bacterial activity. The compounds efficiently suppress drug-resistant HIV-1 strains, and resistance development in cell culture is difficult to engender. Therefore, the (lipophilic) aglycon antibiotic derivatives are important new antiretroviral compounds for the treatment of HIV infections In addition, their early intervention in the infection cycle of HIV also make these compounds potential candidate drugs for prevention of HIV spread [i.e. as a microbicide when given locally (i.e. intravaginally)].

### Example 5

### Cytostatic and Anti-HIV Activity of Some Glycopeptide Antibiotics and Derivatives

| Compound | IC₅₀^{a} (μM) | | | EC₅₀^{b} (μM) | |
|---|---|---|---|---|---|
| No. | L1210 | Molt4/C8 | CEM | HIV-1 | HIV-2 |
| Teicoplanin | >500 | >500 | >500 | 18 ± 3.5 | 100 ± 0 |
| Teicoplanin | >500 | >500 | >500 | 17 ± 3.5 | 20 ± 0 |
| aglycon | | | | | |
| Eremomycin | >500 | >500 | >500 | 50 ± 28 | 250 ± 0.0 |
| aglycon | | | | | |
| Vancomycin | >500 | >500 | >500 | 65 ± 7.1 | 250 ± 0.0 |
| aglycon | | | | | |
| 1 | 53 ± 9 | >100 | >100 | 12 ± 3.5 | 22 ± 3.5 |
| 2 | 60 ± 8 | 53 ± 1 | 172 ± 15 | 5.5 ± 0.7 | >50 |
| 3 | 22 ± 0.3 | 24 ± 18 | 95 ± 14.1 | 5.1 ± 3.3 | 20 |
| 4 | 16 ± 6 | 33 ± 5 | 27 ± 7 | 7.0 ± 0 | >20 |
| 5 | 24 ± 0.4 | 17 ± 3 | 40 ± 4 | 0.43 ± 0.25 | >10 |
| 6 | 250 ± 39 | >500 | >500 | 5.5 ± 0.7 | 12 ± 3.5 |
| 7 | 84 ± 22 | >100 | >100 | 4.0 ± 0 | 3.5 ± 0.7 |
| 8 | >100 | >100 | >100 | 4.0 ± 1.7 | 5.5 ± 0.7 |
| 99 | 94 ± 15 | 126 ± 11 | 148 ± 3 | 1.6 ± 0.36 | 7.0 ± 0.0 |
| 100 | >250 | >250 | >250 | 41.7 ± 20.2 | >125 |
| 101 | >250 | >250 | >250 | 63.3 ± 53.5 | >125 |
| 102 | >250 | >250 | >250 | 7.5 ± 4.8 | 32.5 ± 3.5 |
| 9 | >250 | >250 | >250 | 13 ± 9.9 | 20 ± 7.1 |
| 109 | >250 | >250 | >250 | 7.3 ± 0.58 | 42.5 ± 10.6 |
| 10 | 44 ± 2.9 | 27 ± 14 | 32 ± 5.0 | 4.0 ± 1.4 | >10 |
| 11 | 20 ± 7.5 | 18 ± 2.5 | 80 ± 6.0 | 5.0 ± 0.7 | >10 |
| 12 | 36 ± 14 | 66 ± 20 | >250 | 12 ± 3.5 | >50 |
| 13 | 25 ± 0.7 | 35 ± 6.1 | 212 ± 54 | 3.5 ± 2.1 | 20 |
| 14 | 27 ± 0.3 | 22 ± 4.7 | 92 ± 5.0 | 3.5 ± 0.7 | ≧20 |
| 15 | 48 ± 8 | >100 | >100 | 1.4 ± 0.6 | 6.0 ± 3.9 |
| 16 | 19 ± 5 | 76 ± 8 | 389 ± 99 | 3.5 ± 0.7 | 5.5 ± 2.1 |
| 17 | 97 ± 4.3 | >100 | >100 | 8.0 ± 2.8 | 22 ± 3.5 |
| 18 | 15 ± 2 | 58 ± 11 | 140 ± 26 | 3.0 ± 1.4 | 5.0 ± 1.4 |
| 19 | >500 | >500 | >500 | 2.5 ± 0.7 | 8.0 ± 2.8 |
| 20 | 17 ± 9 | 58 ± 12 | 53 ± 11 | 4.5 ± 0.7 | 44 ± 1.4 |
| 21 | 43 ± 6 | 136 ± 33 | 179 ± 1 | 2.2 ± 0 | 6.5 ± 0.7 |
| 22 | 57 ± 15 | 182 ± 31 | 211 ± 1 | 1.3 ± 0.92 | 7 ± 0 |
| 23 | 5.7 ± 0.27 | 22 ± 22 | 58 ± 35 | 2.6 ± 2.0 | 5.5 ± 0.7 |
| 24 | 12 ± 6 | 41 ± 8 | 46 ± 8 | 4.0 ± 0 | 6.0 ± 0 |
| 25 | 175 ± 44 | 47 ± 4 | 113 ± 28 | 2.1 ± 1.3 | 5.0 ± 0 |
| 26 | 13 ± 0.4 | 36 ± 27 | 228 ± 91 | 5.0 ± 1.4 | 4.0 ± 1.4 |
| 27 | 9.1 ± 0.9 | 28 ± 0.4 | 18 ± 3 | 1.5 ± 0.42 | 2.3 ± 0.21 |
| 28 | 318 ± 256 | >500 | >500 | 3.5 ± 0.7 | 8.5 ± 2.1 |
| 29 | 26 ± 8.1 | 35 ± 8.2 | >250 | 4.5 ± 0.7 | 22 ± 3.5 |
| 30 | 29 ± 7 | 108 ± 79 | >500 | 3.0 ± 0 | 5.0 ± 1.4 |
| 31 | 61 ± 10 | >500 | >500 | 1.7 ± 0.42 | 3.0 ± 1.4 |
| 32 | 23 ± 7 | 35 ± 2 | 90 ± 27 | 5.5 ± 2.1 | 12.5 ± 3.5 |
| 33 | 51 ± 26 | 65 ± 1 | 74 ± 5 | 2.2 ± 0 | 7.5 ± 0.7 |
| 34 | 23 ± 11 | 68 ± 1 | 50 ± 8 | 2.7 ± 1.84 | 4.5 ± 0.7 |
| 35 | 10 ± 3 | 100 | 100 | 1.8 ± 0.49 | 7 ± 0 |
| 36 | 12 ± 0.1 | 73 ± 34 | 100 | 2.1 ± 0.14 | 4.2 ± 2.47 |
| 37 | 12 ± 2 | 19 ± 12 | 9.4 ± 1.9 | 1.6 ± 0.58 | 4.3 ± 0.58 |
| 38 | 51 ± 9 | 91 ± 13 | >100 | 2.1 ± 0.92 | 10 ± 0 |
| 39 | 7.3 ± 0.3 | 14 ± 3 | 14 ± 2 | 1.3 ± 0.21 | 1.3 ± 0.21 |
| 40 | 38.7 ± 3.4 | 32.3 ± | 44 ± 0.42 | 1.5 ± 0.7 | 4.5 ± 2.1 |
| 41 | >500 | >500 | >500 | 15 ± 0 | 17.5 ± 3.5 |
| 42 | 38 ± 1 | 72 ± 6 | 66 ± 2 | 1.8 ± 0.49 | 7 ± 0 |
| 43 | ≧500 | 225 ± 8 | 402 ± 138 | 6.5 ± 0.7 | 12.5 ± 3.5 |
| 44 | >500 | >500 | >500 | 12.5 ± 3.5 | 25 ± 7 |
| 45 | >500 | >500 | >500 | 15 ± 7.1 | 17.5 ± 10.6 |
| 46 | >100 | >100 | >100 | 4 ± 0 | 7 ± 4.2 |
| 47 | 70 ± 23 | >100 | >100 | 6 ± 1 | 12 ± 5.2 |
| 48 | >100 | >100 | >100 | 9.7 ± 9 | 12.3 ± 6.8 |
| 49 | 22 ± 0.1 | 25 ± 0.99 | 104 ± 3.0 | 13 ± 9.9 | 6.0 ± 1.4 |
| 50 | 30 ± 5.7 | 26 ± 6.0 | 123 ± 6.0 | 7.0 ± 4.2 | 6.0 ± 1.4 |
| 51 | 212 ± 54 | >250 | >250 | 5.0 ± 1.4 | 17 ± 3.5 |
| 52 | 202 ± 68 | >250 | >250 | 2.5 ± 0.7 | 3.5 ± 2.1 |
| 160 | 92 ± 5 | 97 ± 10 | 106 ± 0 | 3.3 ± 1.4 | 7.5 ± 0.7 |
| 165 | 240 ± 15 | ≧250 | >250 | 9.0 ± 5.3 | 30.0 ± 7.1 |
| 166 | 91 ± 2 | 112 ± 2 | 125 ± 30 | 1.8 ± 0.58 | 7.0 ± 0.0 |
| 167 | 130 ± 1 | 132 ± 9 | 165 ± 34 | 6.0 ± 2.6 | 10.0 ± 2.8 |
| 53 | 95 ± 10 | 122 ± 13 | 240 ± 13 | 17 ± 3.5 | 11 ± 5.7 |
| 54 | 181 ± 4.0 | >250 | >250 | 17 ± 3.5 | 37 ± 18 |
| 55 | 73 ± 24 | ≧250 | 242 ± 11 | 13 ± 9.9 | 17 ± 3.5 |
| 93 | — | — | 81 | 3.5 | 22 |
| 95 | — | — | 92 | 3.5 | ≧20 |
| 106 | — | — | — | 27 | 30 |
| 107 | — | — | — | 5.0 | 3.5 |
| 108 | — | — | — | 2.8 | 2.0 |
| 161 | — | — | — | 1.8 | 8.2 |
| 162 | — | — | — | 4.5 | 11 |
| 163 | — | — | — | 10 | 15 |
| 164 | — | — | — | 10 | 4.0 |

^{a}IC₅₀, or compound concentration required to inhibit tumor cell proliferation by 50%.
^{b}EC₅₀, or compound concentration required to inhibit HIV-induced giant cell formation in CEM cell cultures by 50%.

### Example 6

### Anti-HIV-1 and -HIV-2 Activity of Several Selected Compounds Against Different HIV-1 and HIV-2 Strains and in Different Cell Lines

| | EC₅₀^{a} (μM) | | | | | | |
|---|---|---|---|---|---|---|---|
| | C8166 | | CEM/0 | | | | |
| Comp | HIV-1 | MOLT4/C8 | HIV-1 | HIV-1 | HIV-2 | HIV-1 | HIV-2 |
| No. | (III_{B}) | HIV-1(HE) | (III_{B}) | (HE)- | (EHO) | (MN) | (RF) |
| 35 | 9.0 ± 4.2 | 7.5 ± 0.7 | 7.5 ± 3.5 | 17 ± 3.5 | 11 ± 1.4 | 12 ± 3.5 | 8.5 ± 2.1 |
| 19 | 9.5 ± 3.5 | 9.0 ± 1.4 | 6.0 ± 1.4 | 12 ± 0.0 | 15 ± 0.0 | 13 ± 2.1 | 9.5 ± 3.5 |
| 30 | ≧5 | 4.5 ± 0.7 | 2.8 ± 0.4 | 5.5 ± 0.7 | 11 ± 1.4 | 7.0 ± 0.0 | 3.7 ± 1.25 |
| 31 | 6.6 ± 3.06 | 3.7 ± 0.35 | 2.8 ± 0.4 | 9.5 ± 3.5 | 6.8 ± 0.35 | 6.5 ± 0.7 | 3.7 ± 1.77 |
| 51 | | 25 ± 5.0 | 5.0 ± 1.4 | 40 ± 0.0 | 13 ± 6.6 | 25 ± 7.1 | 9.0 ± 4.2 |
| 46 | | 6.5 ± 4.3 | 4.0 ± 0 | 35 ± 7.1 | 35 ± 7.1 | 9.0 ± 1.4 | 10 ± 0.0 |
| 6 | | 22 ± 2.9 | 5.5 ± 0.7 | 40 ± 0.0 | 50 ± 0.0 | 20 ± 0.0 | 16 ± 5.7 |

^{a}50% Effective concentration, or compound concentration required to inhibit HIV-induced cytopathicity by 50%.

### Example 7

### Anti-HIV-1 Activity of Several Compounds Against Mutant HIV-1 Strains in CEM Cultures

| | EC₅₀^{a} (μM) | | | | |
|---|---|---|---|---|---|
| Compound No. | HIV-1 III_{B} | Leu-100-Ile | Lys-103-Asn | Tyr-181-Cys | Tyr-188-His |
| 35 | 7.5 ± 3.5 | 12.5 ± 3.5 | 9.0 ± 1.4 | 10 ± 0.0 | 12.5 ± 3.5 |
| 19 | 6.0 ± 1.4 | 11.5 ± 2.1 | 8.5 ± 2.1 | 7.5 ± 3.5 | 11.0 ± 1.4 |
| 30 | 2.8 ± 0.4 | 6.0 ± 1.4 | 7.0 ± 0.0 | 10 ± 0.0 | 7.5 ± 0.7 |
| 31 | 2.8 ± 0.4 | 5.3 ± 2.5 | 6.0 ± 1.4 | 8.5 ± 2.1 | 6.0 ± 1.4 |
| 51 | 5.0 ± 1.4 | 24 ± 6.3 | 12 ± 0.0 | 9.5 ± 2.1 | 11 ± 6.4 |
| 46 | 4.0 ± 0.0 | | 17 ± 4.2 | 8.0 ± 0.0 | |
| 6 | 5.5 ± 0.7 | | 11 ± 1.4 | 10 ± 0.0 | |

^{a}50% Effective concentration or concentration required to protect CEM cells against the cytopathicity of HIV by 50%.

### Example 8

### Evaluation of the Compounds for Their Anti-Viral Activity Against Many Other Virusses (BVDV, HSV, FCV, CMV, VZV, SARS virus, etc.)

Several virusses are inhibited by antibiotics that still contained a sugar moiety. For example the vancomycine derivative, 59 was endowed with a marked anti-VZV activity (EC₅₀: 0.87-0.89 μM) at a concentration that was >50-fold lower than its cytostatic concentration, and 5- to 20-fold lower than its cytotoxic concentration (5 to 7 day assay). Compound 1 showed some antiviral activity against feline and human corona virus (FCV) and SARS virus (EC₅₀: 30-43 μM). As an example, the eremomycin derivatives (3-5 and 60-87), 68, 76, 77 and 81 showed activity (EC₅₀: 0.7-7 μM) against VZV. Compound 5, 63 and 64 were active against FCV in Feline Crandel Kidney cells (FCK) with a selectivity of 5 to 10 and 86 and 87 proved clearly active against BVDV. The teicoplanin derivatives 89 and 90 were for example active against VZV (EC₅₀: 1.1 and 50 μM, respectively).

A variety of lipophylic aglycon derivatives of vancomycine, eremomycin, ristomycin, DA40, and teicoplanin have also been made and tested. The vancomycin type aglycons showed pronounced activity against VZV and FCV (i.e. compounds 6, 7, 8, 98 (VZV) and compounds 5, 7, 9, 13, 99, 100, 101 and 109 (FCV). Compound 98, for example was also endowed with anti-herpes virus activity (EC₅₀ HSV-1 and HSV-2: 24 μM). Teicoplanin aglycons showed also activity against VZV (i.e. 113, 121-128, 137, 143, 145, 146), HSV (i.e. 132 and 146 against both HSV-1 and HSV-2), BVDV (i.e. 126) and FCV (i.e. 125, 157-163, 165-167). All antiviral activities were observed at subtoxic concentrations in the respective cell cultures. Also teicoplanin aglycon derivatives with eliminated amino acids 1 and 3, with a disrupted bond between amino acids 1 and 2, or with a disrupted bond between amino acids 6 and 7 showed activity against FCV. It should be noticed that all compounds that were active against the two wild-type VZV strains, showed also an equal inhibitory effect against two thymidine kinase-deficient (strains 07/1 and YS/R) VZV strains. Further examples of anti-viral activity include the anti-CMV activity of for example compounds 21, 25, 26, 27, 31, 59, 124 and 125.

In conclusion, among the glycopeptide antibiotic derivatives studied, many compounds showed inhibitory activity against several DNA viruses (i.e. herpes simplex virus, cytomegalovirus and varicella-zoster virus) and RNA viruses [i.e. HIV, BVDV (a virus that belongs to the same family as hepatitis C virus), and FCV (a feline corona virus that belongs to the same family as the human SARS corona virus)]. Moreover, most compounds that were found active against FCV were also inhibitory against the SARS virus.

### Example 9

### Anti-HSV Activity of Several Compounds in Cell Culture with Their Cytostatic/Cytotoxic Activity

| | | |
|---|---|---|
| EC₅₀ (μM) | MTC | CC₅₀ |

| Code | HSV-1 | HSV-2 | (μM) | (μM) |
|---|---|---|---|---|
| no. | (KOS) | (G) | (HEL) | (HEL) |
| 40 | 9.6 | 9.6 | ≧5-20 | >50 |
| 88 | 120 | >200 | 50-≧200 | >50 |
| 98 | 24 | 24 | ≧20-200 | >50 |
| 115 | 80 | >16 | ≧20-200 | 50 |
| 132 | 9.6 | 9.6 | ≧5-20 | 40 |
| 145 | 48 | 48 | ≧20-≧50 | >50 |
| 146 | 9.6 | 9.6 | ≧5-50 | >50 |

### Example 10

### Anti-VZV Activity of Several Compounds in Cell Culture with Their Cytostatic/Cytotoxic Activity

| | | |
|---|---|---|
| EC₅₀ (μM) | MTC | CC₅₀ |

| Code | VZV | VZV | (μM) | (μM) |
|---|---|---|---|---|
| no. | (YS) | (OKA) | (HEL) | (HEL) |
| 6 | | 4.2 | ≧20-200 | >50 |
| 7 | 0.87 | 1.0 | ≧20 | >50 |
| 8 | 1.5 | 2.3 | ≧20 | >50 |
| 16 | 2.4 | 2.5 | ≧5-50 | 137 |
| 17 | 4.2 | 4.1 | ≧20-200 | >50 |
| 18 | 2.0 | 2.1 | 5-20 | 94 |
| 20 | 2.3 | 2.0 | ≧5-20 | 169 |
| 21 | 2.0 | 1.3 | 5-50 | 107 |
| 24 | 1.9 | 1.2 | ≧5 | 87 |
| 25 | 0.6 | 1.0 | 20-200 | >50 |
| 27 | 0.8 | 1.0 | ≧2-50 | >200 |
| 28 | 2.0 | 2.5 | ≧5-20 | 105 |
| 31 | 0.85 | 0.75 | 20-50 | >200 |
| 32 | 2.3 | ≧5 | 20 | 30 |
| 33 | 2.4 | ≧5 | 5-50 | ≧200 |
| 35 | 1.5 | 2.2 | 20-100 | >200 |
| 36 | 1.9 | ≧5 | ≧20-50 | >200 |
| 37 | >2 | 1.5 | ≧2-20 | 43 |
| 39 | 0.81 | 0.90 | ≧5 | 170 |
| 40 | 0.21 | 0.29 | ≧5-20 | >50 |
| 41 | 12 | >5 | ≧20-50 | 190 |
| 46 | | 0.81 | 20-80 | >50 |
| 59 | 0.89 | 0.87 | 5-20 | >50 |
| 68 | 0.7 | >2 | 5-50 | >200 |
| 76 | >2 | 2.7 | ≧5-20 | 98 |
| 77 | 5 | 7 | ≧20-50 | 129 |
| 81 | | 2.9 | 20-50 | 43 |
| 89 | | 1.1 | ≧5-200 | >50 |
| 90 | | 50 | ≧200 | >50 |
| 98 | 1.3 | 2.3 | ≧20-200 | >50 |
| 113 | 0.42 | 0.68 | ≧5-20 | >50 |
| 115 | 8.9 | >5 | ≧20-200 | 50 |
| 117 | 2.7 | 2.9 | ≧5-80 | >50 |
| 119 | | 5 | 20-400 | >50 |
| 120 | 10 | 9 | ≧20-200 | >200 |
| 121 | 3.1 | 2.3 | ≧5-20 | 40 |
| 122 | 1.0 | 1.0 | 20-50 | >50 |
| 123 | | 2.8 | ≧5-200 | >50 |
| 124 | 0.22 | 0.34 | ≧5-100 | 71 |
| 125 | 0.25 | 0.32 | ≧2-20 | 44 |
| 126 | 0.3 | 1.0 | >5-50 | >50 |
| 127 | 0.3 | 0.7 | ≧5-20 | 44 |
| 128 | 0.8 | 1.8 | 20 | 48 |
| 132 | | 0.97 | ≧5-20 | 40 |
| 136 | 4.1 | 3.1 | 20-50 | >50 |
| 137 | 0.43 | 0.22 | >5-20 | 98 |
| 140 | | 13 | ≧50-200 | >50 |
| 141 | | 7.1 | 50 | >50 |
| 142 | | 20 | 50 | >50 |
| 143 | 2.6 | 2.8 | ≧20-50 | >50 |
| 145 | 2.8 | 3.9 | ≧20-≧50 | >50 |
| 146 | | 2.5 | ≧5-50 | >50 |
| 169 | | 34 | >200 | 50 |

### Example 11

### Anti-CMV Activity of Several Compounds in Cell Culture with Their Cytostatic/Cytotoxic Activity

| | | |
|---|---|---|
| EC₅₀ (μM) | MTC | CC₅₀ |

| Code | CMV | CMV | (μM) | (μM) |
|---|---|---|---|---|
| no. | AD-169 | Davis | (HEL) | (HEL) |
| 18 | 5 | | 5-20 | 94 |
| 21 | 8 | | 5-50 | 107 |
| 25 | 30 | 20 | 20-200 | >50 |
| 26 | 6.6 | | ≧5-50 | 85 |
| 27 | ≧5 | 3.5 | ≧2-50 | >200 |
| 31 | ≧20 | 10 | 20-50 | >200 |
| 37 | >5 | 3.5 | ≧2-20 | 43 |
| 39 | >2 | 4 | ≧5 | 170 |
| 59 | >5 | 16 | 5-20 | >50 |
| 68 | 20 | >20 | 5-50 | >200 |
| 112 | 32 | | ≧20-200 | >200 |
| 122 | >20 | 20 | 20-50 | >50 |
| 124 | 2.4 | 3.2 | ≧5-100 | 71 |
| 125 | 1.9 | 2.8 | ≧2-20 | 44 |
| 127 | 10 | >5 | ≧5-20 | 44 |
| 146 | >20 | 20 | ≧5-50 | >50 |

### Example 12

### Anti-BVDV and Cytostatic/Cytotoxic Activity of Some Selected Compounds (86, 87 and 126) in Cell Culture

| | EC₅₀ (μM) | MTC |
|---|---|---|
| Code no. | BVDV | (MDBK) |
| 86 | 5.3 | ≧100 |
| 87 | 20 | ≧300 |
| 126 | 12 | 60 |

### Example 13

### Anti-FCV, Anti-SARS Virus and Cytostatic/Cytotoxic Activity of Several Compounds in Cell Culture

| Code | EC₅₀ (μM) | CC₅₀ (μM) | EC₅₀ (μM) | CC₅₀ (μM) |
|---|---|---|---|---|
| no. | FCV | FCK | SARS virus | Vero |
| 1 | 30 | >50 | 43 | >100 |
| 5 | 5.4 | 28 | 13 | 54 |
| 7 | 3.6 | >50 | | |
| 9 | 26 | >50 | | |
| 13 | 5.9 | 30 | | |
| 19 | 18 | >50 | 23 | >100 |
| 28 | >50 | 21 | 16 | >100 |
| 30 | 9.2 | ≧50 | 33 | >100 |
| 31 | >100 | ≧20 | 24 | >100 |
| 41 | 21 | >50 | 44 | >100 |
| 47 | 16 | >50 | | |
| 51 | 15 | ≧100 | 39 | >100 |
| 52 | 14 | >50 | | |
| 53 | 36 | ≧100 | | |
| 54 | 19 | >50 | | |
| 55 | 22 | >100 | | |
| 63 | 3.4 | 15 | | |
| 64 | 8.2 | 75 | 31 | >100 |
| 99 | 23 | ≧100 | | |
| 100 | 20 | >100 | | |
| 101 | 47 | >100 | | |
| 102 | | >100 | 68 | >100 |
| 106 | 74 | >300 | | |
| 107 | 40 | >300 | | |
| 108 | 81 | >300 | | |
| 109 | 28 | >100 | | |
| 124 | | 17 | 15 | >100 |
| 125 | 1.6 | 14 | 10 | >100 |
| 159 | 9.9 | 85 | 28 | >100 |
| 160 | 4.5 | 56 | 27 | >100 |
| 161 | 9.4 | >100 | 34 | >100 |
| 162 | 4.7 | ≧100 | 22 | >100 |
| 163 | 2.2 | 63 | 26 | >100 |
| 165 | 11 | 80 | 26 | >100 |
| 166 | 8.5 | 49 | 31 | >100 |
| 167 | 7.7 | 62 | 47 | >100 |
| 170 | 23 | >100 | 32 | >100 |
| 53 | 36 | ≧100 | 29 | >100 |
| 54 | 19 | >50 | 47 | >100 |
| 171 | | >100 | 29 | >100 |
| 55 | 22 | >100 | 31 | >100 |
| 172 | ≧100 | >100 | 38 | >100 |

1-22. (canceled)

23. A glycopeptide antibiotic or derivative thereof according to formula I, II or III: 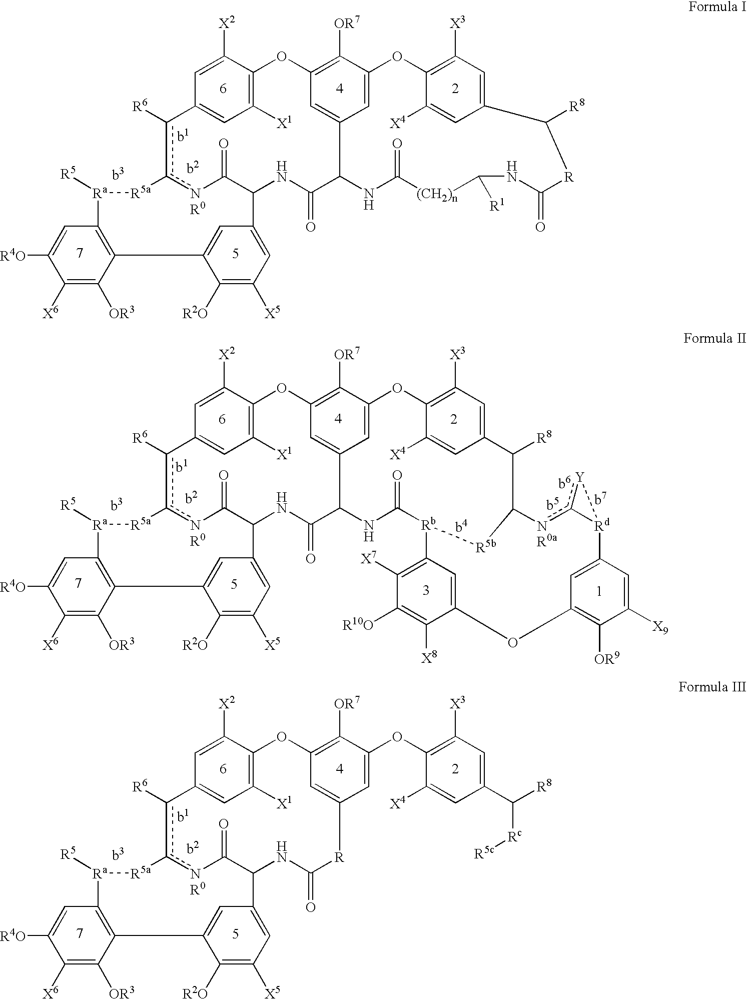
wherein:
each b¹ and b² independently represents nihil or an additional bond, while b¹ and b² can not be an additional bond at the same time, R⁰ represents nihil when b² represents an additional bond and hydrogen when b² represents nihil, R⁶ represents nihil when b¹ represents an additional bond and hydrogen when b¹ represents nihil, R⁶ represents R^{6a} and R⁰ represents hydrogen when b¹ and b² each represents nihil;
b³ represents nihil or an additional bond, R^{a}—R^{5a} represents a group of the formula CHN(R¹¹)CO, CHN(R¹¹)(CH₂)_{z}N(R^{11a})CO or CHN(R¹¹)CO(CH₂)_{z}N(R^{11a})CO when b³ represents an additional bond, and R^{a} is R and R^{5a} is R⁵ when b³ represents nihil, wherein z is 0, 1, 2, 3 or 4;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R^{11a})CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and R^{d} represents R or a group of the formula (CH₂)_{q}CON(R¹¹)CH(CH₂OH)(CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R¹¹) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen, halogen and X⁶;
X⁶ is selected from the group comprising hydrogen, halogen, SO₃H, OH, NO, NO₂, NHNH₂, NHN═CHR¹¹, N═NR¹¹, CHR¹¹R¹³, CH₂N(R³)R¹¹, R⁵, R¹¹ and R¹³, wherein R³ is CH₂ attached to the phenolic hydroxyl group of the 7^{th} amino acid;
X⁸ is selected from hydrogen and alkyl;
R^{c} represents R and R^{5c} represents R⁵;
R is selected from CHR¹³ and R¹⁴;
R¹ is selected from hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)—[O-phenyl(o-OR⁹, m-X⁹, m-R¹⁶)]-m, where t is 0, 1, 2, 3 or 4;
each R² and R⁴ are independently selected from hydrogen, R¹² and R¹⁷;
R³ is selected from hydrogen, R¹², R¹⁷ and Sug;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁷ is selected from hydrogen, R¹², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug.
R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁹ is selected from hydrogen, R¹², R¹⁷ or Sug;
R¹⁰ is selected from hydrogen, R¹², R¹⁷ or Sug, wherein Sug is any cyclic or acyclic carbohydrate;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl, a heterocyclic ring, alkylphosphonate (e.g. alkylenePO₂OH) and alkylphosphonamide unsubstituted or substituted at the amide with alkyl, alkenyl or alkynyl(e.g alkylenePO₂NH₂), wherein each alkyl, alkenyl, alkynyl, aryl, arylalkyl, heteroaryl, cyloalkyl, cycloalkenyl, cycloalkynyl and heterocyclic ring can be substituted with 1 or more R¹⁹ or Sug;
each R¹² and R^{12a} are independently selected from the group consisting of hydrogen, acyl, amino-protecting group, carbamoyl, thiocarbamoyl, SO₂R¹¹, S(O)R¹¹, COR¹³—R¹⁸, COCHR¹⁸N(NO)R¹¹, COCHR¹⁸NR¹¹R¹² and COCHR¹⁸N⁺R¹¹R^{11a}R^{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹³ is selected from the group consisting of hydrogen, NHR^{12a}, NR¹¹R¹², NR¹¹Sug, N⁺R¹¹R^{11a}R^{11b}, R¹⁵, NR¹¹C(R^{11a}R^{11b})COR¹⁵ and group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R¹¹R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R₁₂;
R¹⁵ is selected from N(R¹¹)NR^{11a}R¹², N(R¹¹)OR^{11a}, NR¹¹C(R^{11a}R_{11b})COR¹³;
R¹⁶ is selected from a group of the formula R—R⁵ or CH(NH₂)CH₂OH;
R¹⁷ is selected from SO₃H, SiR¹¹R^{11a}R^{11b}, SiOR¹¹OR^{11a}R^{11b}, PR¹¹R^{11a}, P(O)R¹¹R^{11a}, P⁺R¹¹R^{11a}R^{11b};
R¹⁸ is selected from hydrogen, R¹, alkyl, aryl, phenyl-rhamnose-p, phenyl-(rhamnose-galactose)-p, phenyl-(galactose-galactose)-p, phenyl-O-methylrhamnose-p, wherein each alkyl and aryl can be substituted with 1 or more R¹⁹ or Sug,
R¹⁹ is selected from hydrogen, halogen, SH, SR²⁰, OH, OR²⁰, COOH, COR²⁰, COOR²⁰ NO₂, NH₂, N(R²⁰)₂NHC(NH₂)═NH, CH(NH₂)═NH, NHOH, NHNH₂, N₃, NO, CN, N═NR²⁰, N═NR¹², SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰;
—R²⁰ is selected from hydrogen, halogen, SH, OH, COOH, NO₂, NH₂, NHC(NH₂)═NH, CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring.

24. The glycopeptide antibiotic or derivative thereof according to claim 23, wherein:
each b¹ and b² represent nihil, R⁶ represents R^{6a} and R⁰ represents hydrogen;
b³ represents an additional bond and R^{a}—R^{5a} represents CHNHCO;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R¹¹)CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and R^{d} represents R or a group of the formula (CH₂)_{q}CON(R¹¹)CH(CH₂OH)(CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R¹¹) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen and halogen;
X⁶ is CH₂R¹³;
X⁸ is selected from hydrogen and methyl;
R^{c} represents R and R^{5c} represents R⁵;
R is CHR¹³;
R¹ is selected from the group consisting of hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)—[O-phenyl(o-OR⁹, m-X⁹, m-R¹⁶)]-m, where t is 0, 1, 2, 3 or 4;
each R² and R⁴ are independently selected from hydrogen, R¹² and R¹⁷;
R³ is selected from hydrogen, R¹², R¹⁷, mannosyl and O-acetylmanosyl;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug and Sug is selected from glucosyl, ristosaminyl, N-acetylglucosaminyl, 4-epi-vancosaminyl, 3-epi-vancosaminyl, vancosaminyl, actinosaminyl, glucuronyl, 4-oxovancosaminyl, ureido-4-oxovancosaminyl and their derivatives;
R⁷ is selected from hydrogen, R¹², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug, wherein Sug is selected from glucosyl, mannosyl, ristosaminyl, N-acylglucosaminyl, N-acylglucuronyl, glucosaminyl, glucuronyl, 4-epi-vancosaminyl, 3-epi-vancosaminyl, vancosaminyl, actinosaminyl, acosaminyl, glucosyl-vancosaminyl, glucosyl-4-epi-vancosaminyl, glucosyl-3-epi-vancosaminyl, glucosyl-acosaminyl, glucosyl-ristosaminyl, glucosyl-actinosaminyl, glucosyl-rhamnosyl, glucosyl-olivosyl, glucosyl-mannosyl, glucosyl-4-oxovancosaminyl, glucosyl-ureido-4-oxovancosaminyl, glucosyl(rhamnosyl)-mannosyl-arabinosyl, glucosyl-2-O-Leu and their derivatives.
R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug, wherein Sug is selected from mannosyl, galactosyl and galactosyl-galactosyl;
R⁹ is selected from hydrogen, R¹², R¹⁷, galactosyl and galactosyl-galactosyl;
R¹⁰ is selected from hydrogen, R¹², R¹⁷, mannosyl or fucosyl;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹² is selected from the group consisting of hydrogen, acyl, amino-protecting group, carbamoyl, thiocarbamoyl, SO₂R¹¹, S(O)R¹¹, COR¹³—R¹⁸, COCHR⁸N(NO)R¹¹, COCHR¹⁸NR¹¹R¹² and COCHR¹⁸N⁺R¹¹R^{11a}R^{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R^{12a} is selected from the group consisting of hydrogen, COCHR¹⁸NR¹¹R¹², COCHR¹⁸N(NO)R¹¹, COCHR¹⁸N⁺R¹¹R^{11a}R^{11b} and COCHR¹⁸R¹³;
R¹³ is selected from the group consisting of hydrogen, NHR^{12a}, NR¹¹R², NR¹¹Sug, N⁺R¹¹R^{11a}R^{11b}, R¹⁵, NR¹¹C(R^{11a}R^{11b})COR¹⁵ and a group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R¹¹R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R₁₂;
R¹⁵ is selected from N(R¹¹)NR^{11a}R¹², N(R¹¹)OR^{11a}, NR¹¹C(R^{11a}R^{11b})COR¹³;
R¹⁶ is selected from a group of the formula R—R⁵ or CH(NH₂)CH₂OH;
R¹⁷ is selected from SO₃H, SiR¹¹R^{11a}R^{11b}, SiOR¹¹OR^{11a}OR^{11b}, PR¹¹R^{11a}, P(O)R¹¹R^{11a}, P⁺R¹¹R^{11a}R^{11b};
R¹⁸ is selected from hydrogen, R¹, CH₃, CH₂CH(CH₃)₂, phenyl(p-OH, m-CI), phenyl-rhamnose-p, phenyl-(rhamnose-galactose)-p, phenyl-(galactose-galactose)-p, phenyl-O-methylrhamnose-p;
R¹⁹ is selected from hydrogen, halogen, SH, SR²⁰, OH, COOH, COR²⁰, COOR²⁰NO₂, NH₂, N(R²⁰)₂NHC(NH₂)═NH, CH(NH₂)═NH, NHOH, NHNH₂, N₃, NO, CN, N═NR²⁰, N═NR¹², SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰;
R²⁰ is selected from hydrogen, halogen, SH, OH, COOH, NO₂, NH₂, NHC(NH₂)═NH, CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring.

25. The glycopeptide antibiotic or derivative thereof according to claim 23, wherein the derivative is not a compound of the group of compounds referred to with the codes 1 to 55 in the description of this application.

26. The glycopeptide antibiotic or derivative thereof according to claim 23, selected from the group of compounds referred to with the codes 56 to 172 in the description of this application.

27. A composition containing a glycopeptide antibiotic or derivative thereof according to claim 23 as an active ingredient.

28. A composition for separate, combined or sequential use in the treatment or prophylaxis of anti-viral infections, comprising:
a) one or more compounds according to claim 23, and,
b) one or more compounds effective in the treatment or prophylaxis of viral infections,
including Retroviral, Flaviviral, Herpes or Coronaviral enzyme or entry inhibitors, in proportions such as to provide a synergistic effect in the said treatment or prophylaxis.

29. A method for preventing or treating a viral infections in a subject or patient by administering to the patient in need thereof a therapeutically effective amount of one or more glycopeptide antibiotics or derivatives thereof.

30. The method of claim 29, wherein the one or more glycopeptide antibiotics or derivatives thereof are cyclic glycopeptide antibiotics or derivatives thereof wherein the second amino acid is a phenolic amino acid.

31. The method of claim 29, wherein the one or more glycopeptide antibiotics or derivatives thereof are selected from the group consisting of vancomycin, teicoplanin, eremomycin, chloroeremomycin, dechloroeremomycin, ristomycin or DA40926.

32. The method of claim 29, wherein the one or more glycopeptide antibiotics or derivatives thereof are of the formula I, II, or III, pharmaceutically acceptable salts, solvates, tautomers and isomers thereof, 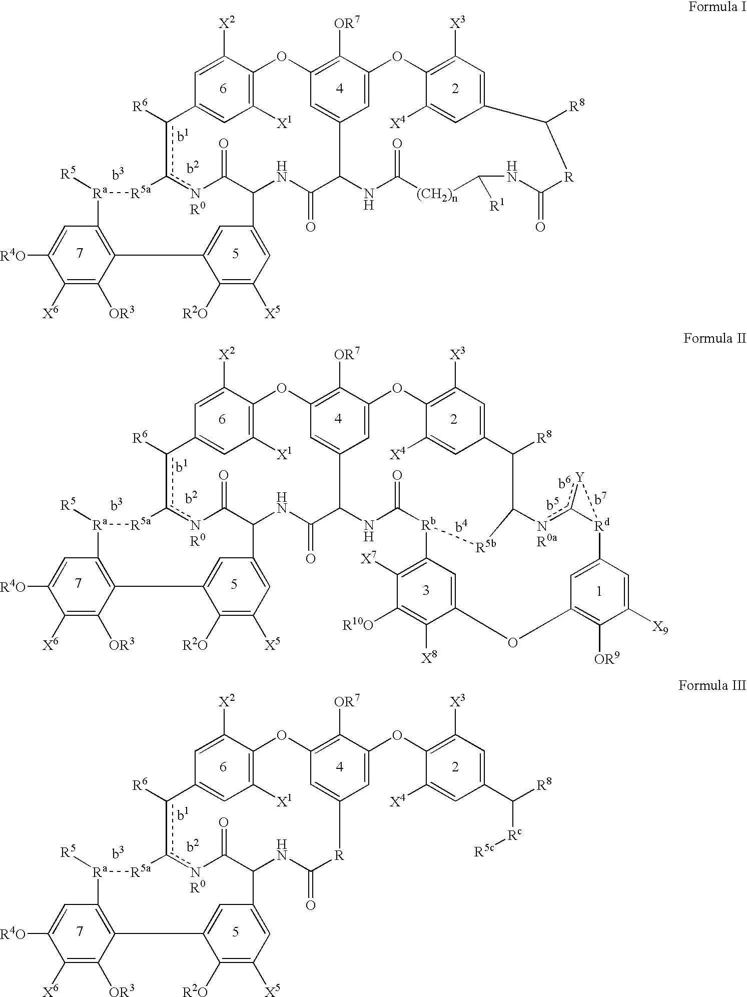
wherein:
each b¹ and b² independently represents nihil or an additional bond, while b¹ and b² can not be an additional bond at the same time, R⁰ represents nihil when b² represents an additional bond and hydrogen when b² represents nihil, R⁶ represents nihil when b¹ represents an additional bond and hydrogen when b¹ represents nihil, R⁶ represents R^{6a} and R⁰ represents hydrogen when b¹ and b² each represents nihil;
b³ represents nihil or an additional bond, R^{a}—R^{5a} represents a group of the formula CHN(R¹¹)CO, CHN(R¹¹)(CH₂)_{z}N(R^{11a})CO or CHN(R¹¹)CO(CH₂)_{z}N(R^{11a})CO when b³ represents an additional bond, and R^{a} is R and R^{5a} is R⁵ when b³ represents nihil, wherein z is 0, 1, 2, 3 or 4;
b⁴ represents nihil or an additional bond, R^{b}—R^{5b} represents a group of the formula CHN(R¹¹)CO, CHN(R¹¹)(CH₂)_{z}N(R^{11a})CO or CHN(R¹¹)CO(CH₂)ₚN(R^{11a})CO when b⁴ represents an additional bond, and R^{b} is R and R^{5b} is R⁵ when b⁴ represents nihil, wherein p is 0, 1, 2, 3 or 4;
each b⁵, b⁶ and b⁷ independently represents nihil or an additional bond; Y represents oxygen, R^{0a} represents hydrogen and Rd represents R or a group of the formula (CH₂)_{q}CON(R¹¹)CH(CH₂OH)(CH₂)_{q}N(R¹²)CH(CH₂OH) when b⁵ and b⁷ represent nihil and b⁶ represents an additional bond. R^{0a} represents nihil, R^{d}—Y represents a group of the formula CHN═C(NR¹¹)O or CHNHCON(R¹¹) when b⁶ represents nihil and b⁵ represents an additional bond. Y and R^{0a} each represents a hydrogen and R^{d} represents group of the formula (CH₂)_{q}CON(R¹¹)CH(CH₂OH)(CH₂)_{q}N(R¹²)CH(CH₂OH) when b⁵, b⁶ and b⁷ each represents nihil, wherein q is 0, 1, 2, or 3 and n is 0, 1, 2 or 3;
each X¹, X², X³, X⁴, X⁵, X⁷ and X⁹ are independently selected from hydrogen, halogen and X⁶;
X⁶ is selected from the group comprising hydrogen, halogen, SO₃H, OH, NO, NO₂, NHNH₂, NHN═CHR¹¹, N═NR¹¹, CHR¹¹R¹³, CH₂N(R³)R¹¹, R⁵, R¹¹ and R¹³, wherein R³ is CH₂ attached to the phenolic hydroxyl group of the 7^{th} amino acid;
X⁸ is selected from hydrogen and alkyl;
R^{c} represents R and R^{5c} represents R⁵;
R is selected from CHR¹³ and R¹⁴;
R¹ is selected from hydrogen, R¹¹, (CH₂)ₜCOOH, (CH₂)ₜCONR¹¹R¹², (CH₂)ₜCOR¹³, (CH₂)ₜCOOR¹¹, COR¹⁵, (CH₂)ₜOH, (CH₂)ₜCN, (CH₂)ₜR¹³, (CH₂)ₜSCH₃, (CH₂)ₜSOCH₃, (CH₂)ₜS(O)₂CH₃, (CH₂)ₜphenyl(m-OH, p-CI), (CH₂)ₜphenyl(o-X⁷, m-OR¹⁰, p-X⁸)—[O-phenyl(o-OR⁹, m-X⁹, m-R¹⁶)]-M, where t is 0, 1, 2, 3 or 4;
each R² and R⁴ are independently selected from hydrogen, R¹² and R¹⁷;
R³ is selected from hydrogen, R¹², R¹⁷ and Sug;
R⁵ is selected from COOH, COOR¹¹, COR¹³, COR¹⁵, CH₂OH, CH₂halogen, CH₂R¹³, CHO, CH═NOR¹¹, CH═NNR¹¹R¹² and C═NNHCONR¹¹R¹²;
R^{6a} is selected from OR¹², OR¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁷ is selected from hydrogen, R², R¹⁷, Sug and alkyl-Sug, alkenyl-Sug, alkynyl-Sug, wherein each alkyl, alkenyl and alkynyl can be unsubstituted or substituted with 1 or more R¹⁹ or Sug.
R⁸ is selected from hydrogen, R¹², R¹⁷, OH, O-alkyl-Sug, O-alkenyl-Sug, O-alkynyl-Sug and O-Sug, wherein each alkyl, alkenyl and alkynyl can be unsubstituted or substituted with 1 or more R¹⁹ or Sug;
R⁹ is selected from hydrogen, R¹², R¹⁷ or Sug;
R¹⁰ is selected from hydrogen, R¹², R¹⁷ or Sug, wherein Sug is any cyclic or acyclic carbohydrate;
each R¹¹, R^{11a} and R^{11b} are independently selected from the group consisting of hydrogen, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
each R¹² and R^{12a} are independently selected from the group consisting of hydrogen, acyl, amino-protecting group, carbamoyl, thiocarbamoyl, SO₂R¹¹, S(O)R¹¹, COR¹³—R¹⁸, COCHR¹⁸N(NO)R¹¹, COCHR¹⁸NR¹¹R¹² and COCHR¹⁸N⁺R¹¹R^{11a}R_{11b}, alkyl, alkenyl, alkynyl, aryl, arylalkyl, heteroaryl, cyloalkyl, cycloalkenyl, cycloalkynyl and a heterocyclic ring, wherein each alkyl, alkenyl, alkynyl, aryl, arylalkyl, heteroaryl, cyloalkyl, cycloalkenyl, cycloalkynyl and a heterocyclic ring can be substituted with 1 or more R¹⁹ or Sug;
R¹³ is selected from the group consisting of hydrogen, NHR^{12a}, NR¹¹R¹², NR¹¹Sug, N⁺R¹¹R^{11a}R^{11b}, R¹⁵, NR¹¹C(R^{11a}R^{11b})COR¹⁵ and group of the formula N-A-N⁺-A, wherein A is —CH₂—B—CH₂— and B is —(CH₂)ₘ-D-(CH₂)ᵣ—, wherein m and r are from 1 to 4 and D is O, S, NR¹², N⁺R¹¹R^{11a};
R¹⁴ is CH₂, C═O, CHOH, C═NOR¹¹, CHNHOR¹¹, C═NNR¹¹R¹², C═NNHCONR¹¹R¹² and CHNHNR¹¹R¹²;
R¹⁵ is selected from N(R¹¹)NR^{11a}R¹², N(R¹¹)OR^{11a}, NR¹¹C(R^{11a}R^{11b})COR¹³;
R¹⁶ is selected from a group of the formula R—R⁵ or CH(NH₂)CH₂OH;
R¹⁷ is selected from SO₃H, SiR¹¹R^{11a}R^{11b}, SiOR¹¹OR^{11a}OR^{11b}, PR¹¹R^{11a}, P(O)R¹¹R^{11a}, P⁺R¹¹R^{11a}R^{11b};
R¹⁸ is selected from hydrogen, R¹, alkyl, aryl, phenyl-rhamnose-p, phenyl-(rhamnose-galactose)-p, phenyl-(galactose-galactose)-p, phenyl-O-methylrhamnose-p, wherein each alkyl and aryl can be substituted with 1 or more R¹⁹ or Sug,
R¹⁹ is selected from hydrogen, halogen, SH, SR²⁰, OH, OR²⁰, COOH, COR²⁰, COOR²⁰ NO₂, NH₂, N(R²⁰)₂NHC(NH₂)═NH, CH(NH₂)═NH, NHOH, NHNH₂, N₃, NO, CN, N═NR²⁰, N═NR¹², SOR²⁰, SO₂R²⁰, PO₂OR²⁰, PO₂N(R²⁰)₂, B(OH)₂, B(OR²⁰)₂, CO, CHO, O-Sug, NR²⁰-Sug, R²⁰, R¹², R¹⁷ and R¹⁸ and each R¹⁹ can be substituted with 1 or more R²⁰;
R²⁰ is selected from hydrogen, halogen, SH, OH, COOH, NO₂, NH₂, NHC(NH₂)═NH, CH(NH₂)═NH, NHOH, NHNH₂, N₃, NO, CN, alkyl, alkenyl, alkynyl, aryl, arylalkyl, heteroaryl, cyloalkyl, cycloalkenyl, cycloalkynyl and a heterocyclic ring.

33. The method according to claim 29, wherein said glycopeptide antibiotic or derivatives thereof are selected from the group consisting of the compounds 1 to 172 in the description of the application.

34. The method according to claim 29, wherein said viral infection is an infection of a virus belonging to the family of the Retroviridae such as HIV.

35. The method according to claim 29, wherein said viral infection is an infection of a virus belonging to the family of the Flaviviridae, the Herpesviridae or the Coronaviridae.

36. The method according to claim 35, wherein said viral infection is an infection with Hepatitis C virus (HCV), the virus causing SARS, Herpes simplex virus (HSV-1 or 2), Cytomegalovirus (CMV), Varicella Zoster virus (VZV), Feline Corona virus (FCV) or Bovine viral diarrhoea virus' (BVDV).

37. A method of screening antiviral compounds which comprises:
a) providing glycopeptide antibiotics or derivatives thereof, and,
b) determining the anti-viral activity of said compound.

38. A method for selecting antiviral glycopeptide antibiotics and derivatives thereof which comprises,
a) providing glycopeptide antibiotics or derivatives thereof, and
b) determining the anti-viral and the anti-bacterial activity and the cell toxicity of said compound, and selecting the compound with the best anti-viral activity, the lowest anti-bacterial activity and the lowest cell toxicity.
